(19) United States
(12) Patent Application Publication
(10) Pub. No.: US 2012/0077959 A1
(43) Pub. Date: Mar. 29, 2012

(54) Asymmetric Cobalt-Catalyzed Cyclopropanation With Succinimidyl Diazoacetate

(76) Inventors: X. Peter Zhang, Tampa, FL (US); Joshua V. Ruppel, Tampa, SC (US)

(21) Appl. No.: 13/201,544

(22) PCT Filed: Feb. 16, 2010
(86) PCT No.: PCT/US10/24299

§ 371 Date: Dec. 12, 2011

Related U.S. Application Data
(60) Provisional application No. 61/152,802, filed on Feb. 16, 2009.

(51) Int. Cl. C07K 5/08; C07D 265/30; C07C 231/02; C07D 207/46; C07C 229/28; C07H 5/06; C07D 207/16

(52) U.S. Cl. 530/331; 536/29.1; 544/176; 548/539; 548/542; 560/41; 564/134

(57) ABSTRACT

Cobalt(II) complexes of the D₂-symmetric chiral porphyrins are effective catalysts for asymmetric cyclopropanation reactions with succinimidyl diazoacetate. The Co-catalyzed reaction is suitable for various olefins, providing the corresponding cyclopropane succinimidyl esters in high yields and excellent diastereo- and enantio-selectivity. The resulting enantioenriched cyclopropane succinimidyl esters can serve as convenient synthons for the general synthesis of optically active cyclopropyl carboxamides through mild reactions with a wide range of amine derivatives, including unprotected peptides and amino sugars

### CROSS-REFERENCE TO RELATED APPLICATIONS

This application claims priority to U.S. Provisional Application Ser. No. 61/152,802, filed Feb. 16, 2009, which is incorporated herein by reference in its entirety.

### STATEMENT OF GOVERNMENT RIGHTS

This invention was made with Government support under grant number NSF 0711024 awarded by the National Science Foundation and grant number 44286-AC1 awarded by the American Chemical Society. The government has certain rights in the invention.

### FIELD OF THE INVENTION

The present invention generally relates to metal-catalyzed cyclopropanation of olefins. More particularly, the present invention relates to a process for asymmetric cyclopropanation of olefins using succinimidyl diazoacetates as a reagent.

### BACKGROUND OF THE INVENTION

The well-documented importance of cyclopropanes in numerous fundamental and practical applications has stimulated vast efforts for the synthesis of these smallest carbocycles. See, e.g., Pietruszka, J. Chem. Rev. 2003, 103, 1051; Wessjohann et al., Chem. Rev. 2003, 103, 1625; Donaldson, Tetrahedron 2001, 57, 8589; and Salaun, J. Chem. Rev. 1989, 89, 1247. Metal-catalyzed asymmetric cyclopropanation of alkenes with diazo reagents constitutes the most direct and general approach for the stereoselective construction of these unique all-carbon triangular structures. See, e.g., Lebel et al., Chem. Rev. 2003, 103, 977; Davies et al., Org. React. 2001, 57, 1; Doyle et al., Chem. Rev. 1998, 98, 911; Padwa et al., Tetrahedron 1992, 48, 5385; and Doyle, Chem. Rev. 1986, 86, 919. A number of outstanding chiral catalysts have been reported to achieve high diastereo- and enantio-selectivity for several classes of cyclopropanation reactions, most of which employed diazoacetates. For select examples of asymmetric cyclopropanation with diazocarbonyls, see Hu et al., Org. Lett. 2002, 4, 901; Niimi et al., Adv. Syn. Catal. 2001, 343, 79; Davies et al., Eur. J. Org. Chem. 1999, 2459; Lo et al., J. Am. Chem. Soc. 1998, 120, 10270; Davies et al., J. Am. Chem. Soc. 1996, 118, 6897; Nishiyama et al., J. Am. Chem. Soc. 1994, 116, 2223; Doyle et al., J. Am. Chem. Soc. 1993, 115, 9968; Evans et al., J. Am. Chem. Soc. 1991, 113, 726; and Fritschi et al., Agnew. Chem., Int. Ed. Engl. 1986, 25, 10051-3. Ongoing endeavours in the field aim at further expanding the substrate scope to include a broader variety of alkenes as well as to utilize more challenging classes of diazo reagents for use in asymmetric cyclopropanation.

In contrast to the large body of excellent results achieved with diazoacetates, diazoacetamides have not been successfully employed for asymmetric intermolecular cyclopropanation (Reaction Scheme A) except for the Rh₂-based intramolecular reactions by Doyle and co-workers.

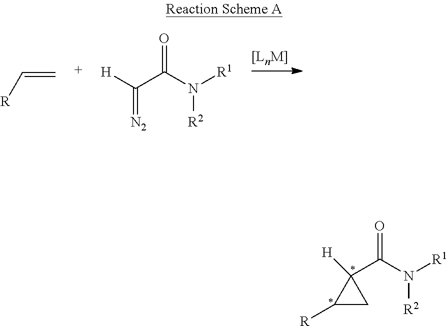

For non-asymmetric cyclopropanation with α-diazoacetamides, see, e.g., Doyle et al., J. Org. Chem. 1985, 50, 1663; Jeganathan et al., J. Org. Chem. 1986, 51, 5362; Doyle et al., Tetrahedron Lett. 1987, 28, 833; Haddad et al., Tetrahedron: Asymmetry 1997, 8, 3367; Gross et al., Tetrahedron Lett. 1999, 40, 1571; and Muthusamy et al., Synlett 2003, 1599. For Rh₂-based intramolecular reactions by Doyle, see Doyle et al., J. Am. Chem. Soc. 1995, 117, 5763; Doyle et al., J. Org. Chem. 1996, 61, 2179; and Doyle et al., Tetrahedron 1994, 50, 1665.

The absence of effective intermolecular asymmetric cyclopropanation with diazoacetamides may be attributed to two major factors:

i) inherent low reactivity of the resulting metal-carbene intermediate due to reduced electrophilicity and increased steric hindrance and ii) complications resulting from competitive intramolecular C—H insertion. For select examples of intramolecular carbene C—H insertion of α-diazoacetamides, see: Brown et al., J. Org. Chem. 1994, 59, 2447; Gois et al., Eur. J. Org. Chem. 2004, 3773; and Grohmann et al., Adv. Synth. Catal. 2006, 348, 2203.

Structurally well-defined cobalt(II) complexes of D₂-symmetric chiral porphyrins ([Co(Por*)]) have emerged as a class of effective catalysts for asymmetric cyclopropanation reactions, with both electron-sufficient and electron-deficient olefins using diazoacetates, diazosulfones, and α-nitro-diazoacetates. See, for example, Chen et al., J. Am. Chem. Soc. 2004, 126, 14718; Chen et al., J. Org. Chem. 2007, 72, 5931; Chen et al., J. Am. Chem. Soc. 2007, 129, 12074; Zhu et al., J. Am. Chem. Soc. 2008, 130, 5042; Zhu et al., Agnew. Chem., Int. Ed. Engl. 2008; and DOI: 10.1002/anie.200803857.

### SUMMARY OF THE INVENTION

Inspired by their important biomedical applications (Garrido et al., J. Bioorg. Med. Chem. Lett. 2006, 16, 1840; Jiang et al., Bioorg. Med. Chem. Lett. 2006, 16, 2105; Sandanayaka et al., J. Med. Chem. 2003, 46, 2569; Morain et al., CNS Drug Rev. 2002, 8, 31; and Graham et al., J. Med. Chem. 1987, 30, 1074) we envisioned a post-derivatization approach to synthesize chiral cyclopropyl carboxamides 2 in enantioenriched form through reacting preformed cyclopropyl chiral building blocks 1 with various amines (Scheme B).

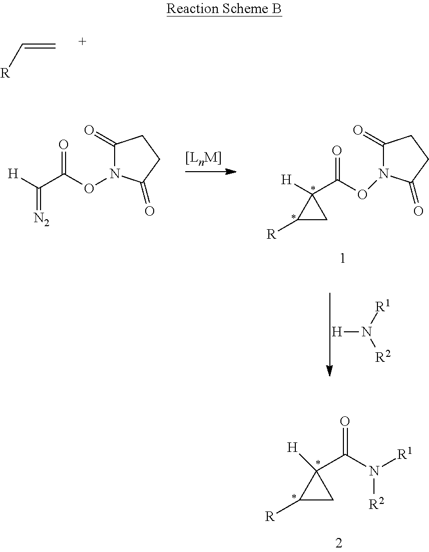

Here, we report a cobalt-catalyzed asymmetric cyclopropanation process with succinimidyl diazoacetate (N₂CHCO₂Su), which results in the formation of cyclopropanes 1 in excellent diastereo- and enantioselectivities. The highly reactive hydroxysuccinimide esters 1, in turn, serve as convenient synthons for the general preparation of chiral amides 2 through reactions with a range of different amines, and without loss of pre-established enantiomeric purity.

Among the various aspects of the present invention, therefore, is the provision of a process for the preparation of chiral cyclopropyl carboxamides in diastereo- and enantioenriched form, the provision of a process which results in the formation of cyclopropanes with high diastereo- and enatioselectivities, and the provision of key intermediates in such processes.

Briefly, therefore, the present invention is directed to A process for the preparation of a chiral cyclopropyl carboxamide in diastereo- and enantioenriched form, the process comprising treating a succinimidyl cyclopropyl carboxylate with an amine, in the presence of a metal porphyrin catalyst.

Another aspect of the invention is a process for the preparation of a chiral cyclopropyl carboxamide in enantioenriched form, the process comprising treating a stereoisomer with an amine in a reaction mixture in the presence of a metal porphyrin catalayst. The stereoisomer corresponds to Formula C-1

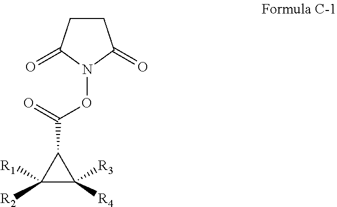

wherein R₁, R₂, R₃ and R₄ are independently hydrogen, hydrocarbyl, substituted hydrocarbyl, heterocyclo, or EWG, and each EWG is independently an electron withdrawing group. In addition, the reaction mixture has an enantiomeric excess of the sterioisomer over its enantiomer.

Another aspect of the present invention is a succinimidyl cyclopropyl carboxylate corresponding to Formula C

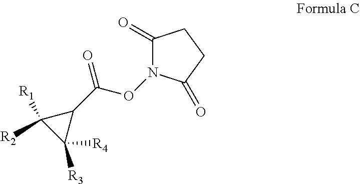

wherein R₁, R₂, R₃ and R₄ are independently hydrogen, hydrocarbyl, substituted hydrocarbyl, heterocyclo, or EWG, and each EWG is independently an electron withdrawing group.

Another aspect of the present invention is an enantioenriched cyclopropyl carboxamide corresponding to Formula CA:

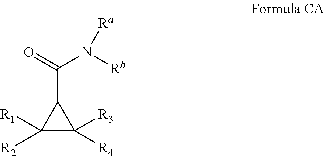

wherein R₁, R₂, R₃, and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo.

Other aspects and features will be in part apparent and in part pointed out hereinafter.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N— or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO— where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “diazo” or “azo” as used herein alone or as part of another group denote an organic compound with two linked nitrogen compounds. These moieties include without limitation diazomethane, ethyl diazoacetate, and t-butyl diazoacetate.

The term “electron acceptor” as used herein denotes a chemical moiety that accepts electrons. Stated differently, an electron acceptor is a chemical moiety that accepts either a fractional electronic charge from an electron donor moiety to form a charge transfer complex, accepts one electron from an electron donor moiety in a reduction-oxidation reaction, or accepts a paired set of electrons from an electron donor moiety to form a covalent bond with the electron donor moiety.

The terms “halogen” or “halo” as used herein alone or as part of another group denote chlorine, bromine, fluorine, and iodine.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The term “heteroatom” as used herein denotes atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainded of the molecule through a carbon or heteroatom. Exemplary heterocyclo include heteroaromatics as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, as alkaryl, alkenaryl, and alkynaryl. Unless otherwise indicated, these moieties preferably comprise 1 to 20 carbon atoms.

The term “porphyrin” as used herein denotes a compound comprising a fundamental skeleton of four pyrrole nuclei united through the α-positions by four methane groups to form the following macrocyclic structure:

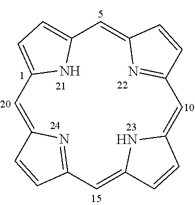

The term “substituted hydrocarbyl” as used herein alone or as part of another group denotes hydrocarbyl moieties which are substituted with at least one atom other than carbon, including moieties in which a carbon chain atom is substituted with a hetero atom as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

One aspect of the present invention is directed to a process for the preparation of a chiral cyclopropyl carboxamide in enantioenriched form. In general, the process comprises treating a succinimidyl cyclopropyl carboxylate with an amine. In one preferred embodiment, the succinimidyl cyclopropyl carboxylate substrate is prepared by a highly diastereo- and enantio-selective Co-catalyzed asymmetric cyclopropanation of alkenes with succinimidyl diazoacetate (N₂CHCO₂Su).

Succinimidyl cyclopropyl carboxylates, in general, and succinimidyl cyclopropyl carboxylates corresponding to Formula C, C-1, C-2, C-3, C-4, C-5 and/or C-6, in particular, may be prepared by treating compounds containing an ethylenic bond, commonly known as olefins with succinimidyl diazoacetate, N₂CHCO₂Su wherein Su is succinimidyl, in the presence of a metal porphyrin complex. Advantageously, the metal porphyrin catalyzed process proceeds relatively efficiently under relatively mild and neutral conditions, in a one-pot fashion, with olefins as limiting reagents.

The preparation of chiral cyclopropyl carboxamide preferably proceeds in two steps. In the first step, an olefin is treated with a succinimidyl diazoacetate to form a cyclopropyl carboxylate. In the second step, the cyclopropyl carboxylate is treated with an amine to form the corresponding cyclopropyl carboxamide. Reaction Scheme 1 illustrates a preferred embodiment of the two steps

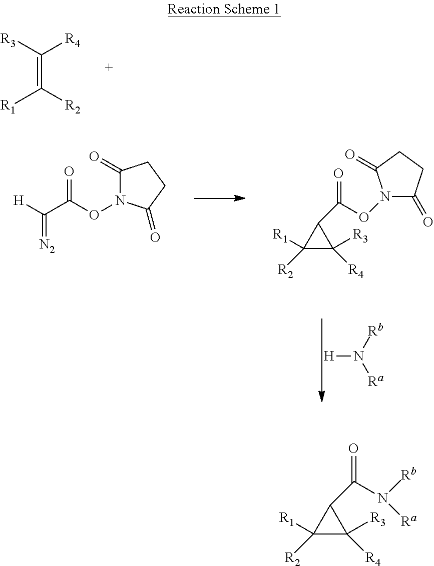

wherein R₁, R₂, R₃ and R₄ are independently hydrogen, hydrocarbyl, substituted hydrocarbyl, heterocyclo, or EWG, each EWG is independently an electron withdrawing group, and R^{a} and R^{b} are independently hydrogen, hydrocarbyl, substituted hydrocarbyl, or heterocyclo. The first step is preferably carried out in the presence of a catalytic amount of a cobalt porphyrin catalyst.

Olefin Substrates

In general, olefins may be used as substrates to form a cyclopropyl carboxylate. In one embodiment, the olefin corresponds to Formula O-1:

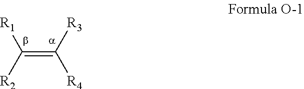

wherein R₁, R₂, R₃, and R₄ are independently hydrogen, hydrocarbyl, substituted hydrocarbyl, heterocyclo, or an electron withdrawing group. For example, in one embodiment, R₁ may be hydrogen. By way of further example, R₁ may be alkyl or substituted alkyl. By way of further example, R₁ may be aryl or substituted aryl. In one embodiment, R₂ is hydrogen. In another embodiment, R₂ is alkyl or substituted alkyl. In yet another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl. For example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, one of R₁, R₂, R₃, and R₄ is an electron withdrawing group. In one embodiment, R₃, R₄ and the α-carbon, or R₁, R₂ and the β-carbon form a carbocyclic or heterocyclic ring. In another embodiment, R₁, R₃, the α-carbon, and the β-carbon or R₂, R₄, the α-carbon, and the β-carbon form a carbocyclic or heterocyclic ring. In another embodiment, R₁, R₄, the α-carbon, and the β-carbon or R₂, R₃, the α-carbon, and the β-carbon form a carbocyclic or heterocyclic ring.

When the olefin corresponds to Formula 1 and one, but only one of R₁, R₂, R₃, and R₄ is an electron withdrawing group, e.g., R₁ is an electron withdrawing group, the olefin corresponds to Formula O-1-EWG:

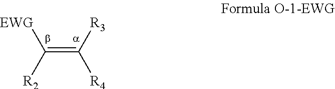

wherein R₁ is an electron withdrawing group and R₂, R₃, and R₄ are as defined in connection with Formula O-1. That is, R₂, R₃, and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, one of R₂, R₃, and R₄ is an electron withdrawing group. In one embodiment, R₃, R₄ and the α-carbon, or R₁, R₂ and the β-carbon form a carbocyclic or heterocyclic ring. In another embodiment, R₁, R₃, the α-carbon, and the β-carbon or R₂, R₄, the α-carbon, and the β-carbon form a carbocyclic or heterocyclic ring. In another embodiment, R₁, R₄, the α-carbon, and the β-carbon or R₂, R₃, the α-carbon, and the β-carbon form a carbocyclic or heterocyclic ring.

When the olefin corresponds to Formula O-1 and R₁ is hydrogen, the olefin corresponds to Formula O-2:

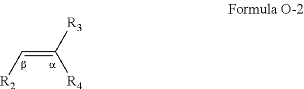

wherein R₂, R₃, and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₃ and R₄ are both hydrogen. In one embodiment, one of R₂, R₃, and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —N⁺O₂ where R is hydrocarbyl, substituted hydrocarbyl or heterocyclo and X is halogen, optionally substituted alkyl or optionally substituted aryl. In one embodiment, R₃, R₄ and the α-carbon form a carbocyclic or heterocyclic ring. In another embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

When the olefin corresponds to Formula O-1, R₁ is hydrogen, and one but only one of R₃ and R₄ is hydrogen, the olefin corresponds to Formula O-3-cis or Formula O-3-trans:

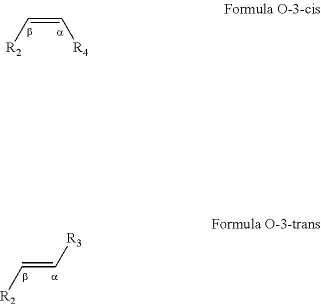

wherein R₂, R₃ and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, one of R₂ and R₃ is an electron withdrawing group. In one embodiment, R₄ is hydrogen. In another embodiment, R₄ is alkyl or substituted alkyl. In one embodiment, one of R₂ and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

When the olefin corresponds to Formula O-1 and R₃ and R₄ are both hydrogen, the olefin is a terminal alkene, corresponding to Formula O-4:

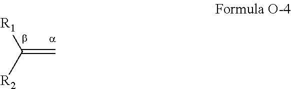

wherein R₁ and R₂ are independently hydrogen, hydrocarbyl, substituted hydrocarbyl, heterocyclo, or an electron withdrawing group. In one embodiment, one but only one of R₁ and R₂ is an electron withdrawing group. In another embodiment, R₁ and R₂ are independently hydrocarbyl, substituted hydrocarbyl, or heterocyclo. In one embodiment, one of R₁ and R₂ is hydrogen. In one embodiment, R₁, R₂, and the β-carbon form a carbocyclic or heterocyclic ring.

When the olefin corresponds to Formula O-1 and R₂, R₃, and R₄ are hydrogen, the olefin is a terminal olefin corresponding to Formula O-5:

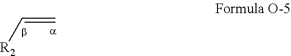

wherein R₁ is hydrogen, hydrocarbyl, substituted hydrocarbyl, heterocyclo, or an electron withdrawing group. In one embodiment, R₂ is alkyl, substituted alkyl, aryl, substituted aryl, or acyl. In one embodiment, R₂ is phenyl or substituted phenyl. In another embodiment, R₂ is alkyl. In another embodiment, R₂ is an electron withdrawing group. In one embodiment, R₂ is phenyl or substituted phenyl. In another embodiment, R₂ is acyl. In another embodiment, R₁ is an electron withdrawing group. In another embodiment, R₂ is R₂₂C(O)— wherein R₂₂ is alkyl, substituted alkyl, alkoxy, or amino.

In general, the olefin's electron withdrawing group(s), EWG, as depicted in Formula O-1-EWG and described in connection with Formula O-1, Formula O-2, Formula O-3-trans, Formula O-3-cis, Formula O-4 or Formula O-5, is any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Rₐ” and “R_{b}”, Rₐ and R_{b} may independently be hydrogen or alkyl.

In accordance with one preferred embodiment, the electron withdrawing group(s) is/are a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, C(X)₃, —CN, —SO₃H, —N⁺H₃, —N⁺R₃, or —NO₂⁺ where R is hydrocarbyl, substituted hydrocarbyl or heterocyclo and X is halogen.

As illustrated more fully in the examples, the diastereo- and enantio-selectivity can be influenced, at least in part, by selection of the metal porphyrin complex. Similarly, stereoselectivity of the reaction may also be influenced by the selection of chiral porphyrin ligands with desired electronic, steric, and chiral environments. Accordingly, the catalytic system of the present invention may advantageously be used to control stereoselectivity.

Cyclopropyl Carboxylates

In one embodiment, the cyclopropyl carboxylates produced by the reaction of the olefin and the succinimidyl diazoacetate correspond to Formula C:

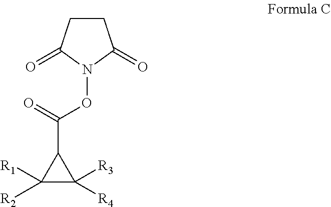

wherein R₁, R₂, R₃, and R₄ are independently hydrogen, hydrocarbyl, substituted hydrocarbyl, heterocyclo, or an electron withdrawing group. In one embodiment, R₁ is hydrogen. In another embodiment, R₁ is alkyl or substituted alkyl. In another embodiment, R₁ is aryl or substituted aryl. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are both hydrogen. In one embodiment, one of R₁, R₂, R₃, and R₄ is an electron withdrawing group. In one embodiment, R₃, R₄ and the cyclopropyl ring carbon atom to which they are attached, or R₁, R₂ and the cyclopropyl ring carbon atom to which they are attached, form a carbocyclic or heterocyclic ring. In another embodiment, R₁, R₃, and the cyclopropyl ring carbon atoms to which they are attached, or R₂, R₄, and the cyclopropyl ring carbon atoms to which they are attached, form a carbocyclic or heterocyclic ring. In another embodiment, R₁, R₄, the α-carbon, and the β-carbon or R₂, R₃, the α-carbon, and the β-carbon form a carbocyclic or heterocyclic ring.

In a preferred embodiment, the cyclopropyl carboxylates produced by the reaction of the olefin and the succinimidyl diazoacetate is a stereoisomer corresponding to Formula C-1, the stereoisomer having an enantiomer and the reaction producing an enantiomeric excess of the sterioisomer corresponding to Formula C-1 over its enantiomer

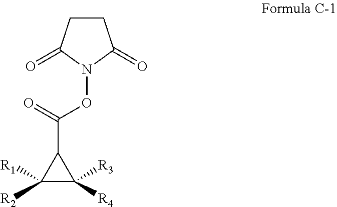

wherein R₁, R₂, R₃, and R₄ are independently hydrogen, hydrocarbyl, substituted hydrocarbyl, heterocyclo, or an electron withdrawing group. In one embodiment, R₁ is hydrogen. In another embodiment, R₁ is alkyl or substituted alkyl. In another embodiment, R₁ is aryl or substituted aryl. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are both hydrogen. In one embodiment, one of R₁, R₂, R₃, and R₄ is an electron withdrawing group. In one embodiment, R₃, R₄ and the and the cyclopropyl ring carbon atom to which they are attached, or R₁, R₂ and the cyclopropyl ring carbon atom to which they are attached form a carbocyclic or heterocyclic ring. In another embodiment, R₁, R₃, and the cyclopropyl ring carbon atoms to which they are attached, or R₂, R₄, and the cyclopropyl ring carbon atoms to which they are attached form a carbocyclic or heterocyclic ring. In another embodiment, R₁, R₄, and the cyclopropyl ring carbon atoms to which they are attached, or R₂, R₃, and the cyclopropyl ring carbon atoms to which they are attached form a carbocyclic or heterocyclic ring.

In one embodiment, the cyclopropyl carboxylates produced by the reaction of the olefin and the succinimidyl diazoacetate is a stereoisomer corresponding to Formula C-2, the stereoisomer having an enantiomer and the reaction producing an enantiomeric excess of the sterioisomer corresponding to Formula C-2 over the enantiomer

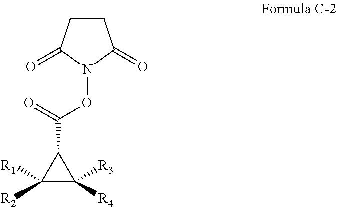

wherein R₁, R₂, R₃, and R₄ are independently hydrogen, hydrocarbyl, substituted hydrocarbyl, heterocyclo, or an electron withdrawing group. In one embodiment, R₁ is hydrogen. In another embodiment, R₁ is alkyl or substituted alkyl. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b wherein R, R}ₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are hydrogen. In one embodiment, one of R₁, R₂, R₃, and R₄ is an electron withdrawing group. In one embodiment, R₃, R₄ and the cyclopropyl ring carbon atom to which they are attached, or R₁, R₂ and the cyclopropyl ring carbon atoms to which they are attached form a carbocyclic or heterocyclic ring. In another embodiment, R₁, R₃, and the cyclopropyl ring carbon atoms to which they are attached, or R₂, R₄, and the cyclopropyl ring carbon atoms to which they are attached form a carbocyclic or heterocyclic ring. In another embodiment, R₁, R₄, and the cyclopropyl ring carbon atoms to which they are attached, or R₂, R₃, and the cyclopropyl ring carbon atoms to which they are attached form a carbocyclic or heterocyclic ring.

In one embodiment, the cyclopropyl carboxylates produced by the reaction of the olefin and the succinimidyl diazoacetate is a stereoisomer corresponding to Formula C-3, the stereoisomer having an enantiomer and the reaction producing an enantiomeric excess of the sterioisomer corresponding to Formula C-3 over the enantiomer

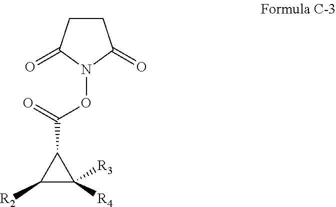

wherein R₂, R₃, and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₃ and R₄ are both hydrogen. In one embodiment, one of R₂, R₃, and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₃, R₄ and the cyclopropyl ring carbon atom to which they are attached form a carbocyclic or heterocyclic ring. In another embodiment, R₂, R₄, and the cyclopropyl ring carbon atoms to which they are attached form a carbocyclic or heterocyclic ring. In another embodiment, R₂, R₃, and the cyclopropyl ring carbon atoms to which they are attached form a carbocyclic or heterocyclic ring.

In one embodiment, the cyclopropyl carboxylates produced by the reaction of the olefin and the succinimidyl diazoacetate is a stereoisomer corresponding to Formula C-4, the stereoisomer having an enantiomer and the reaction producing an enantiomeric excess of the sterioisomer corresponding to Formula C-4 over the enantiomer

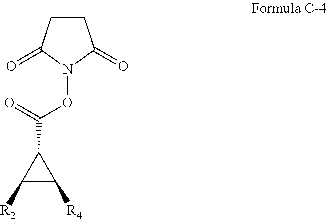

wherein R₂ and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ, and R_{b} are independently optionally substituted alkyl or optionally substituted aryl. In one embodiment, R₄ is hydrogen. In another embodiment, R₄ is alkyl or substituted alkyl. In one embodiment, one of R₂ and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂, R₄, and the cyclopropyl ring carbon atoms to which they are attached form a carbocyclic or heterocyclic ring.

In one embodiment, the cyclopropyl carboxylates produced by the reaction of the olefin and the succinimidyl diazoacetate is a stereoisomer corresponding to Formula C-5, the stereoisomer having an enantiomer and the reaction producing an enantiomeric excess of the sterioisomer corresponding to Formula C-5 over the enantiomer

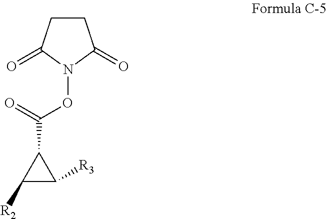

wherein R₂ and R₃ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, one of R₂ and R₃ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂, R₃, and the cyclopropyl ring carbon atoms to which they are attached form a carbocyclic or heterocyclic ring.

In one preferred embodiment, the cyclopropyl carboxylates produced by the reaction of the olefin and the succinimidyl diazoacetate is a stereoisomer corresponding to Formula C-6, the stereoisomer having an enantiomer and the reaction producing an enantiomeric excess of the sterioisomer corresponding to Formula C-6 over the enantiomer

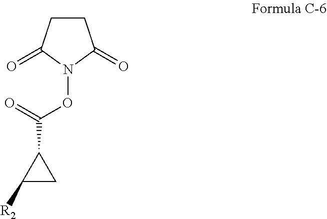

wherein R₂ is hydrocarbyl, substituted hydrocarbyl, heterocyclo, or an electron withdrawing group. In one embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl.

In each of the foregoing embodiments in which the cyclopropyl carboxylate corresponds to Formula C, C-1, C-2, C-3, C-4, C-5 or C-6, it is generally preferred that the enanatiomeric excess of the stereoisomer over its enantiomer be at least 60%. More preferably, the cyclopropyl carboxylate corresponds to Formula C, C-1, C-2, C-3, C-4, C-5 or C-6 and the enanatiomeric excess of the stereoisomer over its enantiomer is at least 80%. Still more preferably, the cyclopropyl carboxylate corresponds to Formula C, C-1, C-2, C-3, C-4, C-5 or C-6 and the enanatiomeric excess of the stereoisomer over its enantiomer is at least 90%. In certain embodiments, the cyclopropyl carboxylate corresponds to Formula C, C-1, C-2, C-3, C-4, C-5 or C-6 and the enanatiomeric excess of the stereoisomer over its enantiomer is at least 95%.

In any of the foregoing embodiments in which the cyclopropyl carboxylate corresponds to Formula C-3, C-4, C-5 or C-6, it is generally preferred that the ratio of the stereoisomer to its diastereomer (i.e., when the carboxylate substituent and R₂ are in the cis conformation rather than the trans conformation as depicted in C-3, C-4, C-5 and C-6) it is generally preferred that the stereoisomer to diasteromer ratio (i.e., the trans:cis ratio) be greater than 90:1, more preferably greater than 98:1 and still more preferably at least 99:1, respectively.

Metal Porphyrins

The porphyrin with which the transition metal is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety).

In a preferred embodiment, the porphyrin is complexed with cobalt. The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In one embodiment, the metal porphyrin complex is a cobalt(II) porphyrin complex. In one particularly preferred embodiment, the cobalt porphyrin complex is a chiral porphyrin complex corresponding to the following structure:

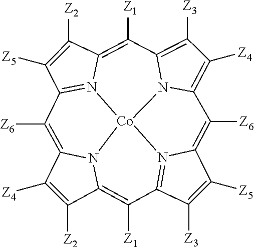

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, Z₆ is substituted phenyl, and Z₁ and Z₆ are different. In one particularly preferred embodiment, Z₂, Z₃, Z₄ and Z₅ are hydrogen, Z₁ is substituted phenyl, Z₆ is substituted phenyl, Z₁ and Z₆ are different, and the porphyrin is a chiral porphyrin. In one even further preferred embodiment, Z₂, Z₃, Z₄ and Z₅ are hydrogen, Z₁ is substituted phenyl, Z₆ is substituted phenyl, Z₁ and Z₆ are different and the porphyrin has D₂-symmetry.

In a preferred embodiment, Z₁ is selected from the group consisting of

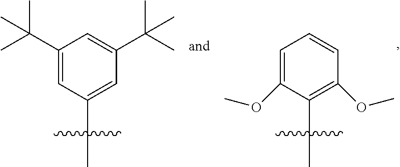

wherein

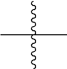

denotes the point of attachment to the porphyrin complex.

In a preferred embodiment, Z₆ is selected from the group consisting of

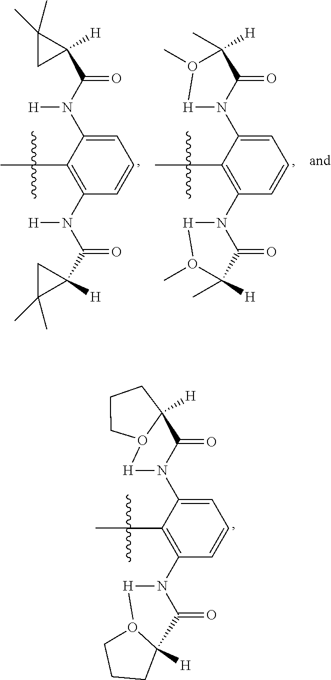

wherein

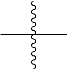

denotes the point of attachment to the porphyrin complex.

Exemplary cobalt (II) porphyrins include the following, designated [Co(P1)], [Co(P2)], [Co(P3)], [Co(P4)], [Co(P5)], and [Co(P6)]:

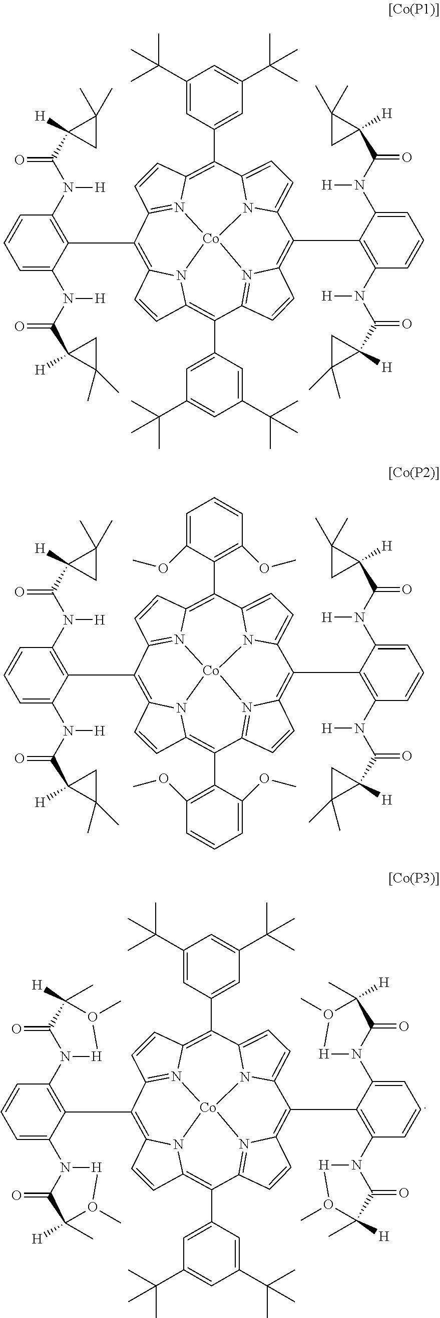

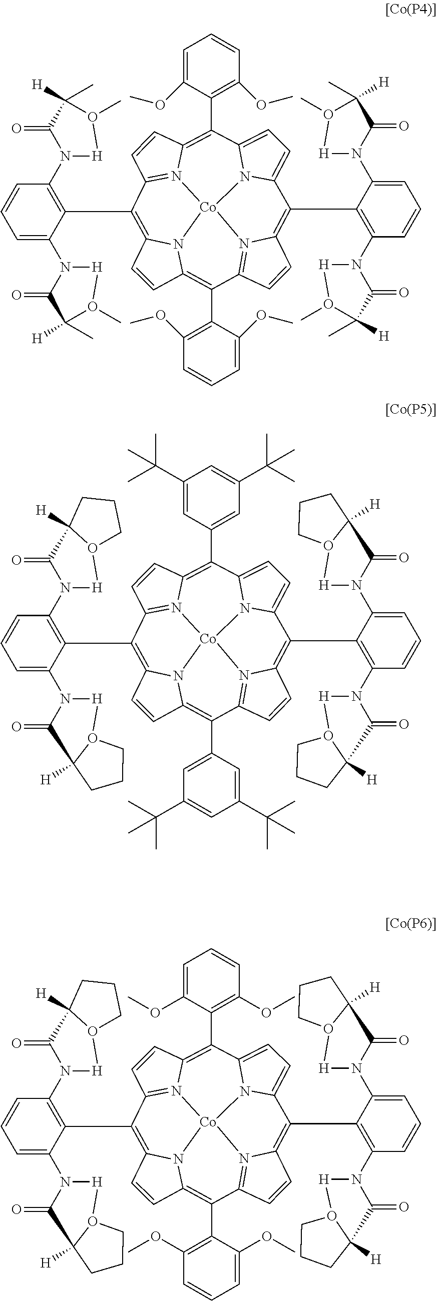

Amines

In general, the cyclopropyl carboxylates of the present invention may be converted to the cyclopropyl carboxamides by treatment with an amine. Typically, the amine will be ammonia, or any of a range of primary or secondary amines that are compatible with the cyclopropyl carboxylate substrate.

In one preferred embodiment, the amine corresponds to the formula HNR^{a}R^{b} wherein R^{a} and R^{b} are independently hydrogen, hydrocarbyl, substituted hydrocarbyl, or heterocyclo. In one embodiment, R^{a} is hydrogen. In another embodiment, R^{b} is hydrogen. In another embodiment, R^{a} is hydrogen and R^{b} is optionally substituted alkyl or optionally substituted aryl. In another embodiment, R^{a} and R^{b} are independently optionally substituted alkyl or optionally substituted aryl. In another embodiment, R^{a} is hydrogen and R^{b} is heterocyclo. In another embodiment, R^{a} is optionally substituted hydrocarbyl and R^{b} is heterocyclo. In another embodiment, R^{a} is optionally substituted alkyl or aryl and R^{b} is heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino.

Cyclopropyl Carboxamides

In one embodiment, the cyclopropyl carboxamides produced by the reaction of the cyclopropyl carboxylate and the amine correspond to Formula CA:

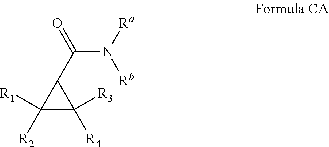

wherein R₁, R₂, R₃, and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In another embodiment, R^{a} and R^{b} are independently optionally substituted alkyl or optionally substituted aryl. In another embodiment, R^{a} is hydrogen and R^{b} is heterocyclo. In another embodiment, R^{a} is optionally substituted hydrocarbyl and R^{b} is heterocyclo. In another embodiment, R^{a} is optionally substituted alkyl or aryl and R^{b} is heterocyclo. In one embodiment, R^{a} is hydrogen and R^{b} is alkyl, substituted alkyl, aryl, substituted aryl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₁, R₂, R₃, and R₄ may be substituted, individually or in combination, as described in connection with the cyclopropyl carboxylate corresponding to Formula C. Thus, for example, in one embodiment R₁ is hydrogen. In another embodiment, R₁ is alkyl or substituted alkyl. In one embodiment, R₂ is hydrogen. In another embodiment, R₁ is alkyl or substituted alkyl. In another embodiment, R₁ is aryl or substituted aryl. In one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are both hydrogen. In one embodiment, one of R₁, R₂, R₃, and R₄ is an electron withdrawing group. In one embodiment, R₃, R₄ and the cyclopropyl ring carbon atom to which they are attached, or R₁, R₂ and the cyclopropyl ring carbon atom to which they are attached, form a carbocyclic or heterocyclic ring. In another embodiment, R₁, R₃, and the cyclopropyl ring carbon atoms to which they are attached, or R₂, R₄, and the cyclopropyl ring carbon atoms to which they are attached, form a carbocyclic or heterocyclic ring. In another embodiment, R₁, R₄, the α-carbon, and the β-carbon or R₂, R₃, the α-carbon, and the β-carbon form a carbocyclic or heterocyclic ring.

In a preferred embodiment, the cyclopropyl carboxamide produced by the reaction of the cyclopropyl carboxylate and the amine is a stereoisomer corresponding to Formula CA-1, the stereoisomer having an enantiomer and the reaction producing an enantiomeric excess of the sterioisomer corresponding to Formula CA-1 over its enantiomer

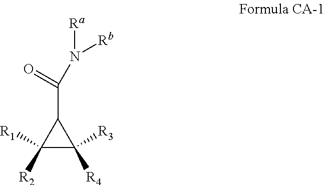

wherein R₁, R₂, R₃, and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₁, R₂, R₃, and R₄ may be substituted, individually or in combination, as described in connection with the cyclopropyl carboxylate corresponding to Formula C-1. Thus, for example, in one embodiment R₁ is hydrogen. In another embodiment, R₁ is alkyl or substituted alkyl. In another embodiment, R₁ is aryl or substituted aryl. In one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are both hydrogen. In one embodiment, one of R₁, R₂, R₃, and R₄ is an electron withdrawing group. In one embodiment, R₃, R₄ and the and the cyclopropyl ring carbon atom to which they are attached, or R₁, R₂ and the cyclopropyl ring carbon atom to which they are attached form a carbocyclic or heterocyclic ring. In another embodiment, R₁, R₃, and the cyclopropyl ring carbon atoms to which they are attached, or R₂, R₄, and the cyclopropyl ring carbon atoms to which they are attached form a carbocyclic or heterocyclic ring. In another embodiment, R₁, R₄, and the cyclopropyl ring carbon atoms to which they are attached, or R₂, R₃, and the cyclopropyl ring carbon atoms to which they are attached form a carbocyclic or heterocyclic ring.

In one embodiment, the cyclopropyl carboxamide produced by the reaction of the cyclopropyl carboxylate and the amine is a stereoisomer corresponding to Formula CA-2, the stereoisomer having an enantiomer and the reaction producing an enantiomeric excess of the sterioisomer corresponding to Formula CA-2 over the enantiomer

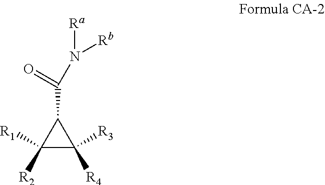

wherein R₁, R₂, R₃, and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₁, R₂, R₃, and R₄ may be substituted, individually or in combination, as described in connection with the cyclopropyl carboxylate corresponding to Formula CA-2. Thus, for example, in one embodiment R₁ is hydrogen. In another embodiment, R₁ is alkyl or substituted alkyl. In one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, R₃ and R₄ are hydrogen. In one embodiment, one of R₁, R₂, R₃, and R₄ is an electron withdrawing group. In one embodiment, R₃, R₄ and the cyclopropyl ring carbon atom to which they are attached, or R₁, R₂ and the cyclopropyl ring carbon atoms to which they are attached form a carbocyclic or heterocyclic ring. In another embodiment, R₁, R₃, and the cyclopropyl ring carbon atoms to which they are attached, or R₂, R₄, and the cyclopropyl ring carbon atoms to which they are attached form a carbocyclic or heterocyclic ring. In another embodiment, R₁, R₄, and the cyclopropyl ring carbon atoms to which they are attached, or R₂, R₃, and the cyclopropyl ring carbon atoms to which they are attached form a carbocyclic or heterocyclic ring.

In one embodiment, the cyclopropyl carboxamide produced by the reaction of the cyclopropyl carboxylate and the amine is a stereoisomer corresponding to Formula CA-3, the stereoisomer having an enantiomer and the reaction producing an enantiomeric excess of the sterioisomer corresponding to Formula CA-3 over the enantiomer

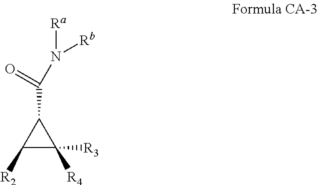

wherein R₂, R₃, and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₁, R₂, R₃, and R₄ may be substituted, individually or in combination, as described in connection with the cyclopropyl carboxylate corresponding to Formula C-3. Thus, for example, in one embodiment R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₃ and R₄ are both hydrogen. In one embodiment, one of R₂, R₃, and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₃, R₄ and the cyclopropyl ring carbon atom to which they are attached form a carbocyclic or heterocyclic ring. In another embodiment, R₂, R₄, and the cyclopropyl ring carbon atoms to which they are attached form a carbocyclic or heterocyclic ring. In another embodiment, R₂, R₃, and the cyclopropyl ring carbon atoms to which they are attached form a carbocyclic or heterocyclic ring.

In one embodiment, the cyclopropyl carboxamide produced by the reaction of the cyclopropyl carboxylate and the amine is a stereoisomer corresponding to Formula CA-4, the stereoisomer having an enantiomer and the reaction producing an enantiomeric excess of the sterioisomer corresponding to Formula CA-4 over the enantiomer

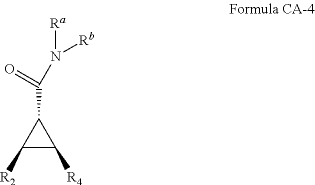

wherein R₂ and R₄ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₂ and R₄ may be substituted, individually or in combination, as described in connection with the cyclopropyl carboxylate corresponding to Formula C-4. Thus, for example, in one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₄ is hydrogen. In another embodiment, R₄ is alkyl or substituted alkyl. In one embodiment, one of R₂ and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂, R₄, and the cyclopropyl ring carbon atoms to which they are attached form a carbocyclic or heterocyclic ring.

In one embodiment, the cyclopropyl carboxamide produced by the reaction of the cyclopropyl carboxylate and the amine is a stereoisomer corresponding to Formula CA-5, the stereoisomer having an enantiomer and the reaction producing an enantiomeric excess of the sterioisomer corresponding to Formula CA-5 over the enantiomer

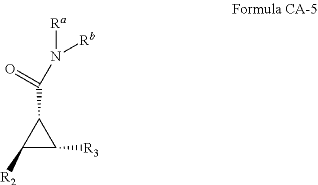

wherein R₂ and R₃ are independently hydrogen, hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₂ and R₃ may be substituted, individually or in combination, as described in connection with the cyclopropyl carboxylate corresponding to Formula C-5. Thus, for example, in one embodiment, R₂ is hydrogen. In another embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, one of R₂ and R₃ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂, R₃, and the cyclopropyl ring carbon atoms to which they are attached form a carbocyclic or heterocyclic ring.

In one preferred embodiment, the cyclopropyl carboxamide produced by the reaction of the cyclopropyl carboxylate and the amine is a stereoisomer corresponding to Formula CA-6, the stereoisomer having an enantiomer and the reaction producing an enantiomeric excess of the sterioisomer corresponding to Formula CA-6 over the enantiomer

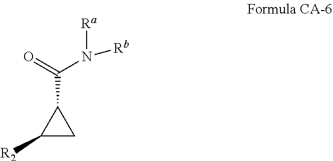

wherein R₂ is hydrocarbyl, substituted hydrocarbyl, heterocyclo, or an electron withdrawing group, and R^{a} and R^{b} are independently hydrogen, hydrocarbyl, substituted hydrocarbyl, or heterocyclo. In one embodiment, R^{a} is hydrogen and R^{b} is alkyl, substituted alkyl, aryl, substituted aryl, or heterocyclo. In one such embodiment, R^{a} is hydrogen and R^{b} is the residue of or comprises a naturally occurring or synthetic α, β, γ, or Δ amino acid or a sugar. For example, in one such embodiment, R^{a} is hydrogen and R^{b} is the residue of an amino acid, a polypeptide, the residue of a sugar, a polysaccaride, an amino sugar, or a nucleotide sugar. By way of further example, in one such embodiment, R^{a} is hydrogen and R^{b} is or comprises the residue of an amino acid selected from the group consisting of isoleucine, leucine, lysine, methionine, phenylalanine, threonine, tyrptophan, valine, alanine, asparagine, aspartic acid, cysteine, glutamic acid, glutamine glycine, proline, selenocysteine, serine, tyrosine, arginine or histidine. By way of further example, in one such embodiment, R^{a} is hydrogen and R^{b} is or comprises the residue of a sugar such as glucose, sucrose, lactose, fructose, galactose, mannose, fusose, or sialic acid. By way of further example, in one such embodiment, R^{a} is hydrogen and R^{b} is or comprises the residue of an amino sugar such as galactosamine, glucosamine, N-acetylgalactosamine, or N-acetylglucosamine. By way of further example, in one such embodiment, R^{a} is hydrogen and R^{b} is or comprises the residue of a nucleotide sugar such as UDP-galactose, UDP-galactosamine, UDP-glucose, UDP-glucosamine, UDP-N-acetylgalactosamine, UDP-N-acetylglucosamine, GDP-mannose, GDP-fusose, CMP-sialic acid, or CMP-N-acetylneuraminic acid. By way of further example, In another embodiment, R^{a} and R^{b} and the nitrogen atom to which they are attached form a heterocyclo, as morpholino or pyrrolidino. In each of the foregoing embodiments, R₂ may be substituted as described in connection with the cyclopropyl carboxylate corresponding to Formula C-6. Thus, for example, in one embodiment, R₂ is alkyl or substituted alkyl. In another embodiment, R₂ is aryl or substituted aryl. For example, in one embodiment, R₂ is phenyl or substituted phenyl wherein the phenyl substituents are selected from the group consisting of alkyl, alkoxy, halo, trihalomethyl, acyloxy, and nitro. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —NO₂⁺ where R is hydrocarbyl, substituted hydrocarbyl or heterocyclo and X is halogen, optionally substituted alkyl or optionally substituted aryl. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl.

In each of the foregoing embodiments in which the cyclopropyl carboxamide corresponds to Formula CA, CA-1, CA-2, CA-3, CA-4, CA-5 or CA-6, it is generally preferred that the enanatiomeric excess of the stereoisomer over its enantiomer be at least 60%. More preferably, the cyclopropyl carboxamide corresponds to Formula CA, CA-1, CA-2, CA-3, CA-4, CA-5 or CA-6 and the enanatiomeric excess of the stereoisomer over its enantiomer is at least 80%. Still more preferably, the cyclopropyl carboxamide corresponds to Formula CA, CA-1, CA-2, CA-3, CA-4, CA-5 or CA-6 and the enanatiomeric excess of the stereoisomer over its enantiomer is at least 90%. In certain embodiments, the cyclopropyl carboxamide corresponds to Formula CA, CA-1, CA-2, CA-3, CA-4, CA-5 or CA-6, and the enanatiomeric excess of the stereoisomer over its enantiomer is at least 95%.

In any of the foregoing embodiments in which the cyclopropyl carboxamide corresponds to Formula CA, CA-1, CA-2, C-3, C-4, C-5 or C-6, it is generally preferred that the ratio of the stereoisomer to its diastereomer (i.e., when the carboxamide substituent and R₂ are in the cis conformation rather than the trans conformation as depicted in CA, CA-1, CA-2, C-3, C-4, C-5 and C-6) it is generally preferred that the stereoisomer to diasteromer ratio (i.e., the trans:cis ratio) be greater than 60:1, more preferably greater than 80:1. In certain embodiments in which the cyclopropyl carboxamide corresponds to Formula CA, CA-1, CA-2, C-3, C-4, C-5 or C-6, it is generally preferred that the ratio of the stereoisomer to its diastereomer be greater than 90:1, more preferably greater than 98:1, still more preferably at least 99:1 and still more preferably at least 99:1, respectively.

Having described the invention in detail, it will be apparent that modifications and variations are possible without departing the scope of the invention defined in the appended claims. Furthermore, it should be appreciated that all examples in the present disclosure are provided as non-limiting examples.

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

General Considerations. Cyclopropanation reactions were performed under nitrogen in oven-dried glassware following standard Schlenk techniques. Toluene was distilled under nitrogen from sodium benzophenone ketyl prior to use. Succinimidyl diazoacetate was synthesized using reported literature procedure. See, for example, Blankley et al., Organic Syntheses; Wiley: New York, 1973; Collect. Vol. V, p 258.; Doyle et al., J. Org. Chem. 1996, 61, 2179.; and Ouihia et al., J. Org. Chem. 1993, 58, 1641. Olefins were purchased from commercial sources and used without further purification. Thin layer chromatography was performed on Merck TLC plates (silica gel 60 F254). Flash column chromatography was performed with Merck silica gel (60 Å, 230-400 mesh, 32-63 μm). ¹H NMR and ¹³C NMR were recorded on a Varian Inova400 (400 MHz) with chemical shifts reported relative to residual solvent. Infrared spectra were measured with a Nicolet Avatar 320 spectrometer with a Smart Miracle accessory. HRMS data was obtained on an Agilent 1100 LC/MS/TOF mass spectrometer. HPLC measurements were carried out on a Shimadzu Prominence LC-20AT HPLC system with a SPD-N20A diode array detector. Enantiomeric excess was measured using either a Chiralcel OD-H or Chiralcel AD-H chiral HPLC column. Optical rotation was measured on a Rudolf Autopol IV polarimeter.

General Procedure for Cyclopropanation. An oven dried Schlenk tube, previously evacuated and backfilled with nitrogen gas, was charged with succinimidyl diazoacetate (0.37 mmol) and catalyst (0.0125 mmol). The Schlenk tube was then evacuated and back filled with nitrogen. The Teflon screw cap was replaced with a rubber septum and a 0.2 ml portion of solvent was added followed by styrene (0.25 mmol), and the remaining solvent (total 1 mL). The Schlenk tube was then purged with nitrogen for one minute and the rubber septum was replaced with a Teflon screw cap. The Schlenk tube was then placed in an oil bath for the desired time and temperature. Following completion of the reaction, the reaction mixture was purified by flash chromatography (hexanes:ethyl acetate=1:1). The fractions containing product were collected and concentrated by rotary evaporation to afford the compound. In most cases, the product was visualized on TLC using the cerium ammonium molybdate (CAM) stain.

As a practical attribute of [Co(Por)]-catalyzed cyclopropanation (see Huang et al., J. Org. Chem. 2003, 68, 8179), these reactions were carried out in a one-pot fashion with styrene as limiting reagent, and without the occurrence of the common dimerization side reaction. Upon examination of the results (Table 1), it was evident that the steric bulkiness of the carbene source governed the reactivity difference of these catalysts. For example, no reactions were observed with the more sterically demanding catalysts [Co(P4)], [Co(P5)], and [Co(P6)] (Table 1, entries 4-6). Furthermore, the yields of the desired cyclopropane 1a by the less steric catalysts [Co(P1)], [Co(P2)], and [Co(P3)] were correlated well with the relative hindrance of the ligand environment (Table 1, entries 1-3). For these reactions, outstanding diastereoselectivities were achieved, with trans-1a produced as the sole diastereomer. While the best ee was attained by [Co(P2)], the use of [Co(P1)] afforded the best yield in addition to high enantioselectivity. Reduction of the N₂CHCO₂Su from 1.5 to 1.2 equivalents gave similarly high diastereo- and enantioselectivity for the [Co(P1)]-catalyzed reaction, but resulted in decreased yields (Table 1, entries 1 and 7). As demonstrated previously (see Chen et al., Synthesis 2006, 1697) a more positive trans effect of DMAP on enantioselectivity was observed (Table 1, entries 7-9). Although selectivity was not affected, by lowering catalyst loading or reducing reaction time, decrease in the overall product yield was observed (Table 1, entries 10 and 11). Finally, toluene seemed to be the solvent of choice as the use of other solvents as chlorobenzene led to lower yields and decreased enantioselectivities (Table 1, entry 12).

TABLE 1
Asymmetric Cycloproponation of Styrene with
Succinimidyl Diazoacetate by D2-
Symmetric Chiral Cobalt(II) Porphyrins.^{a}

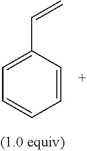

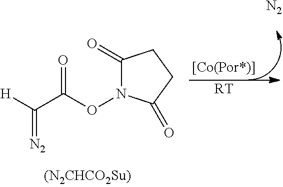

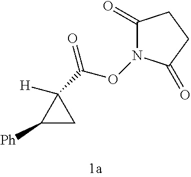

| | | | | yield | trans: | ee |
|---|---|---|---|---|---|---|
| entry | [Co(Por*)]^{b} | additive | solvent | (%) | cis^{a} | (%)^{e} |
| 1 | [Co(P1)] | DMAP | toluene | 86 | >99:1 | 92 |
| 2 | [Co(P2)] | DMAP | toluene | 70 | >99:1 | 96 |
| 3 | [Co(P3)] | DMAP | toluene | 10 | >99:1 | 63 |
| 4 | [Co(P4)] | DMAP | toluene | 0 | | |
| 5 | [Co(P5)] | DMAP | toluene | 0 | | |
| 6 | [Co(P6)] | DMAP | toluene | 0 | | |
| 7 | [Co(P1)] | DMAP | toluene | 74 | >99:1 | 91 |
| 8 | [Co(P1)] | NMI | toluene | 85 | >99:1 | 88 |
| 9 | [Co(P1)] | — | toluene | 86 | >99:1 | 88 |
| 10 | [Co(P1)] | DMAP | toluene | 66 | >99:1 | 91 |
| 11 | [Co(P1)] | DMAP | toluene | 64 | >99:1 | 91 |
| 12 | [Co(P1)] | DMAP | chloro- | 67 | >99:1 | 87 |
| | | | benzene | | | |

^{a}Performed at RT for 48 h using 5 mol % [Co(Por*)] under N₂ with 1.0 equiv of styrene and 1.5 equiv of N₂CHCO₂Su in the presence of 0.5 equiv of DMAP; [styrene] = 0.25M.
^{b}See structures [Co(P1)]-[Co(P6)} above.
^{c}Isolated yields.
^{d}Determined by HPLC.
^{e}Trans isomer ee determined by chiral HPLC.
^{f}1.2 equiv of N₂CHCO₂Suc.
^{g}24 h.
^{h}2 mol %.

Under the optimized reaction conditions, different olefin substrates were subject to catalytic cyclopropanation using N₂CHCO₂Su. As shown with select examples (Table 2), both electron-sufficient and electron-deficient olefins could be successfully cyclopropanated by [Co(P1)]. For example, asymmetric cyclopropanation of styrene derivatives bearing various substituents, including alkyl and halide groups as well as electron-donating and -withdrawing groups, could be catalyzed by [Co(P1)] to form the corresponding cyclopropanes 1a-f in good yields with outstanding diastereoselectivities and excellent enantioselectivities (Table 2, entries 1, 3, 5, 7, 9 and 11). Further improvement in enantioselectivity was achieved uniformly for all these substrates when the relatively bulkier [Co(P2)] was employed as the catalyst, albeit in lower yields for most of the cases (Table 2, entries 2, 4, 6, 8, 10, and 12). In addition, the Co-based catalytic process exhibited functional group tolerance as demonstrated with the reactions of acetoxy- and nitro-substituted styrenes to form 1g-h (Table 2, entries 13 and 14). Due to the steric bulkiness of N₂CHCO₂Su, the catalytic system was shown to be less efficient for large aromatic olefins as exemplified by the [Co(P1)]-catalyzed cyclopropanation reaction of 2-vinylnaphthalene, offering 11 in low yield (Table 2, entry 15). In addition to aromatic olefins, the [Co(P1)]/N₂CHCO₂Su-based system could also selectively cyclopropanate challenging electron-deficient olefins as α,β-unsaturated esters, amides, and ketones (Table 2, entries 16-18). It is worth noting that the cyclopropanes prepared from these olefins (1j-l) are highly electrophilic in nature and have proven to be valuable synthetic intermediates for a variety of applications. See, for example, Gnad et al., Chem. Rev. 2003, 103, 1603; Cativiela et al., Tetrahedron: Asymmetry 2000, 11, 645; Wong et al., Chem. Rev. 1989, 89, 165; and Danishefsky, Acc. Chem. Res. 1979, 12, 66.

With the established availability of enantioenriched succinimidyl cyclopropyl carboxylate derivatives 1 through the [Co(P1)]-catalyzed asymmetric cyclopropanation with N₂CHCO₂Su, their potential application as chiral building blocks for the synthesis of cyclopropyl carboxamides 2 (Scheme 1) was subsequently explored. Using [1R,2R]-1a as a representative synthon, a range of different amines were examined for the post-derivatization synthetic approach (Table 3). Both aliphatic and aromatic amines reacted with 1a smoothly, affording the desired cyclopropyl carboxamides 2a with retention of configuration (Table 3, 2aa and 2ab). Cyclic amines, as pyrrolidine and morpholine, could also be effectively converted to the corresponding amides in high yields with complete preservation of the stereochemistry (Table 3, 2ac and 2ad). The transformation of 1a into the corresponding primary amide using ammonia also occurred in a high yield without loss of diastereo- and enantio-selectivity (Table 3, 2ae). Owing to the mild and neutral reaction conditions, the post-derivatization approach was able to tolerate a number of different functional groups as exemplified by the reactions with chiral α-amino acids as methyl (S)-phenylalaninate as well as chiral β-amino alcohols as (S)-phenylalaminol and (R)-valinol (Table 3, 2af, 2ag, and 2ah). The resulting multi-functional cyclopropyl amides 2af, 2ag, and 2ah, bearing three stereogenic centers, could be isolated as single diastereomers in good to excellent yields.

TABLE 2
[Co(P1)]-Catalyzed Diastereo- and Enantioselective Cyclopropanation of
Different Alkenes with N₂CHCO₂Su.^{a}

| entry | cyclopropane | yield (%)^{b} | trans:cis^{c} | ee (%)^{d} | (α)^{e} |
|---|---|---|---|---|---|
| 1 | 1a | 86 | >99:1 | 92 | (—) |
| 2^{f} | 1a | 70 | >99:1 | 96 | (—) |
| 3 | 1b | 90 | >99:1 | 95 | (—)^{g} |
| 4^{f} | 1b | 71 | 98:2 | 96 | (—)^{g} |
| 5 | 1c | 80 | >99:1 | 97 | (—) |
| 6^{f} | 1c | 81 | >99:1 | 98 | (—) |
| 7 | 1d | 71 | >99:1 | 95 | (—) |
| 8^{f} | 1d | 75 | 99:1 | 97 | (—) |
| 9 | 1e | 66 | >99:1 | 90 | (—) |
| 10^{f} | 1e | 48 | >99:1 | 92 | (—) |
| 11 | 1f | 77 | >99:1 | 90 | (—) |
| 12^{f} | 1f | 30 | >99:1 | 94 | (—) |
| 13 | 1g | 71 | >99:1 | 91 | (—) |
| 14 | 1h | 50 | >99:1 | 92 | (—) |
| 15 | 1i | 33 | 99:1 | 91 | (—) |
| 16 | 1j | 57 | >99:1 | 89 | (—) |
| 17 | 1k | 52 | >99:1 | 96 | (—) |
| 18 | 1l | 55 | >99:1 | 91 | (—) |

^{a}Performed at RT for 48 h using 5 mol % [Co(P1)] under N₂ with 1.0 equiv of styrene and 1.5 equiv of N₂CHCO₂Su in the presence of 0.5 equiv of DMAP; [styrene] = 0.25M.
^{b}Isolated yields.
^{c}Trans:Cis ratio determined by NMR or HPLC.
^{d}Trans isomer ee determined by chiral HPLC.
^{e}Sign of optical rotation.
^{f}[Co(P2)] as catalyst.
^{g}[1R,2R] absolute configuration by X-ray crystal structural analysis and optical rotation.

To further demonstrate the utility of this synthetic approach, [1R,2R]-1a was allowed to react with the unprotected tripeptide [S]-H₂N-Gly-Gly-Ala-COOH at room temperature in a mixture of water and THF in the presence of Et₃N (Equation 1). The corresponding cyclopropyl tripeptide [1R,2R,3S]-2ai was isolated as single diastereomer in 60% yield without affecting the carboxylic acid functionality.

TABLE 3
Post-Derivatization Approach for Synthesis of Chiral Cyclopropyl
Carboxamides via Reaction with Different Amines.^{a}

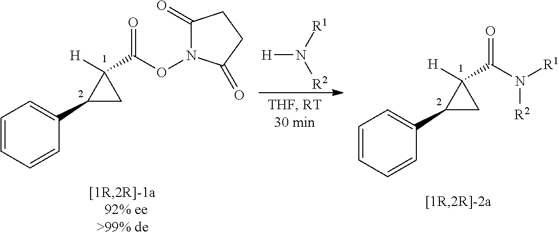

| | |
|---|---|
| 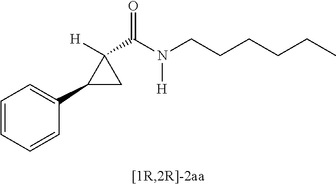 | 92% yield 90% ee >99% de |
| 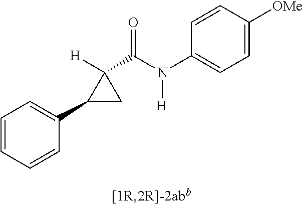 | 62% yield 89% ee >99% de |
| 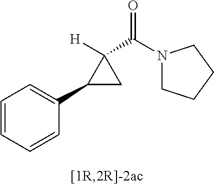 | 91% yield 92% ee >99% de |
| 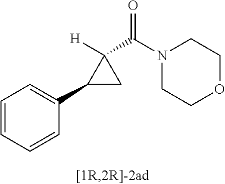 | 95% yield 92% ee >99% de |
| 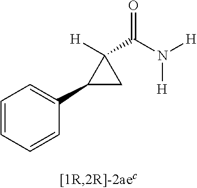 | 93% yield 92% ee >99% de |
| 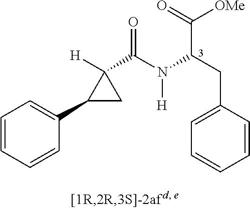 | 66% yield^{f} |
| 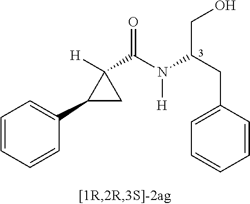 | 93% yield^{f} |
| 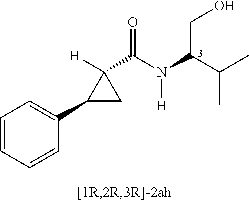 | 54% yield^{f} |

^{a}Isolated yields; de determined by NMR or HPLC; ee determined by chiral HPLC.
^{b}24 h.
^{c}In dioxane.
d1 h.
^{e}In THF/H₂O with Et₃N.
^{f}Isolated as single diastereomer.

The versatility and functional group tolerance of the synthetic approach was further highlighted with the reaction of [1R,21R]-1a with D-(+)-glucosamine without protecting the hydroxyl groups (Equation 2). The reaction proceeded smoothly under mild conditions, forming the desired cyclopropyl carboxamide of the amino sugar [1R,2R,D]-2aj in 47% yield.

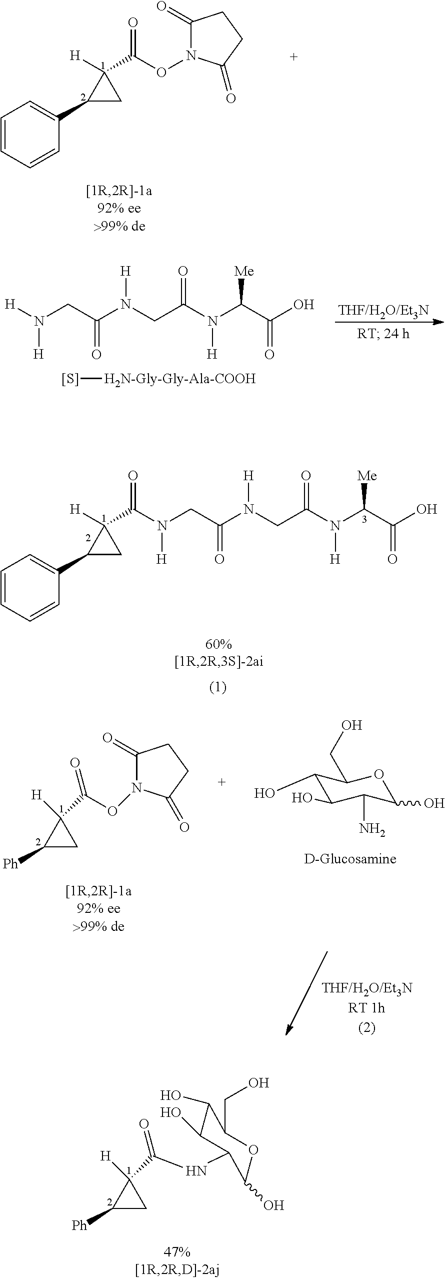

In summary, a highly diastereo- and enantio-selective Co-catalyzed asymmetric cyclopropanation of alkenes with N₂CHCO₂Su has been established for the first time, and the resulting enantioenriched succinimidyl cyclopropylcarboxylates have proven to be valuable synthons for general synthesis of optically active cyclopropyl carboxamide derivatives. The key attributes of the post-derivatization approach include versatility and a high degree of functional group tolerance. Together with the suitability of various olefins for the asymmetric cyclopropanation process, this two-step synthetic scheme should permit straightforward access to a wide range of chiral cyclopropyl carboxamides.

### SUPPORTING EXAMPLES AND DETAILS

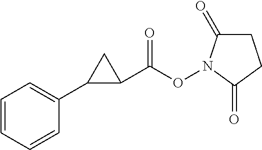

2,5-dioxopyrrolidin-1-yl 2-phenylcyclopropanecarboxylate (1a) was obtained using the general procedure in 86% yield (56.0 mg). [α]²⁰_{D}=−235 (c=0.83, CHCl₃). ¹H NMR (400 MHz, CDCl₃): δ 7.31-7.22 (m, 3H), 7.13-7.11 (m, 2H), 2.82 (bs, 4H), 2.75-2.70 (m, 1H), 2.15-2.11 (m, 1H), 1.80-1.75 (m, 1H), 1.61-1.56 (m, 1H). ¹³C NMR (100 MHz, CDCl₃): δ 169.0, 168.7, 138.3, 128.6, 127.1, 126.3, 28.23, 25.57, 20.87, 18.37. IR (neat, cm⁻¹): 2980 (C—H), 2890 (C—H), 1800 (C═O), 1773 (C═O), 1732 (C═O). HRMS (ESI): Calcd. for C₁₄H₁₃NO₄Na ([M+Na]⁺) m/z 282.07368. Found 282.07304. HPLC Chiralcel OD-H (80 hexanes:20 isopropanol @ 0.8 ml/min): 92% ee; 25 min (minor) and 29 min (major).

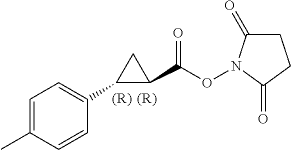

2,5-dioxopyrrolidin-1-yl 2-p-tolylcyclopropanecarboxylate (1b) was obtained using the general procedure in 90% yield (61.7 mg). [α]²⁰_{D}=−296 (c=1.00, CHCl₃). ¹H NMR (400 MHz, CDCl₃): δ 7.11 (d, J=7.8 Hz, 2H), 7.03 (d, J=7.8 Hz, 2H), 2.82 (bs, 4H), 2.74-2.69 (m, 1H), 2.32 (s, 3H), 2.12-2.08 (m, 1H), 1.78-1.73 (m, 1H), 1.59-1.54 (m, 1H). ¹³C NMR (100 MHz, CDCl₃): δ 169.1, 168.7, 136.7, 135.2, 129.2, 126.2, 28.03, 25.53, 20.78, 20.77, 18.23. IR (neat, cm⁻¹): 2924 (C—H), 1783 (C═O), 1735 (C═O). HRMS (ESI): Calcd. for C₁₅H₁₉N₂O₄ ([M+NH₄]⁺) m/z 291.13393. Found 291.13339. HPLC Chiralcel OD-H (80 hexanes:20 isopropanol @ 0.8 ml/min): 95% ee; 20 min (minor) and 26 min (major).

The X-ray intensities were measured using Bruker-APEX2 area-detector CCD diffractometer (CuKa, λ=1.54178 A). Indexing was performed using APEX2. Frames were integrated with SAINT V7.51A software package. Absorption correction was performed by multi-scan method implemented in SADABS. The structure was solved using SHELXS-97 and refined using SHELXL-97 contained in SHELXTL v6.10 and WinGX v1.70.01 programs packages. The X-ray Crystal data and refinement conditions are shown in Table S1.

TABLE S1
Crystal data and structure refinement for
2,5-dioxopyrrolidin-1-yl 2-p-tolylcyclopropanecarboxylate 1b.

| | | |
|---|---|---|
| Empirical formula | C15H15NO4 | |
| Formula weight | 273.28 | |
| Temperature | 296(2) K | |
| Wavelength | 1.54178 Å | |
| Crystal system | Orthorhombic | |
| Space group | P2(1)2(1)2(1) | |
| Unit cell dimensions | a = 5.8676(2) Å | α = 90°. |
| | b = 8.9556(3) Å | β = 90°. |
| | c = 27.5262(8) Å | γ = 90°. |

| | |
|---|---|
| Volume | 1446.44(8) Å³ |
| Z | 4 |
| Density (calculated) | 1.255 Mg/m³ |
| Absorption coefficient | 0.760 mm⁻¹ |
| F(000) | 576 |
| Crystal size | 0.35 × 0.20 × 0.08 mm³ |
| Theta range for data collection | 3.21 to 67.78°. |
| Index ranges | −6 <= h <= 6, −9 <= k <= 10, |
| | −33 <= l <= 31 |
| Reflections collected | 9269 |
| Independent reflections | 1501 [R(int) = 0.0274] |
| Completeness to theta = 67.78° | 96.3% |
| Absorption correction | Semi-empirical from equivalents |
| Max. and min. transmission | 0.9417 and 0.7769 |
| Refinement method | Full-matrix least-squares on F² |
| Data/restraints/parameters | 1501/0/182 |
| Goodness-of-fit on F² | 1.084 |
| Final R indices [I > 2sigma(I)] | R1 = 0.0392, wR2 = 0.1048 |
| R indices (all data) | R1 = 0.0520, wR2 = 0.1103 |
| Absolute structure parameter | 10(10) |
| Largest diff. peak and hole | 0.102 and −0.144 e · Å⁻³ |

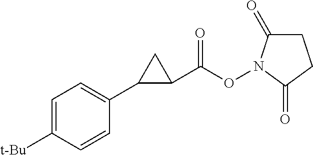

2,5-dioxopyrrolidin-1-yl 2-(4-tert-butylphenyl)cyclo-propanecarboxylate (1c) was obtained using the general procedure in 80% yield (62.8 mg). [α]²⁰_{D}=−269 (c=0.59, CHCl₃). ¹H NMR (400 MHz, CDCl₃): δ 7.34 (d, J=8.0 Hz, 2H), 7.09 (d, J=8.0 Hz, 2H), 2.83 (bs, 4H), 2.74-2.70 (m, 1H), 2.15-2.11 (m, 1H), 1.80-1.75 (m, 1H), 1.62-1.57 (m, 1H), 1.31 (s, 9H). ¹³C NMR (100 MHz, CDCl₃): δ 169.0, 168.7, 150.1, 135.3, 126.0, 125.5, 34.43, 31.26, 27.97, 25.53, 20.84, 18.25. IR (neat, cm⁻¹): 2980 (C—H), 1800 (C═O), 1771 (C═O), 1733 (C═O). HRMS (ESI): Calcd. for C₁₈H₂₁NO₄ ([M+Na]⁺) m/z 338.13628. Found 338.13648. HPLC: Chiralcel OD-H (95 hexanes:5 isopropanol @ 0.8 ml/min): 97% ee; 45 min (minor) and 50 min (major).

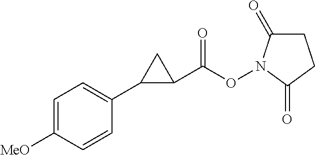

2,5-dioxopyrrolidin-1-yl 2-(4-methoxyphenyl)cyclo-propanecarboxylate (1d) was obtained using the general procedure in 71% yield (51.8 mg). [α]²⁰_{D}=−300 (c=0.59, CHCl₃). ¹H NMR (400 MHz, CDCl₃): δ 7.07 (d, J=8.4 Hz, 2H), 6.84 (d, J=8.8 Hz, 2H), 3.79 (s, 3H), 2.81 (bs, 4H), 2.73-2.68 (m, 1H), 2.08-2.04 (m, 1H), 1.77-1.72 (m, 1H), 1.57-1.52 (m, 1H). ¹³C NMR (100 MHz, CDCl₃): δ 169.1, 168.8, 158.7, 130.2, 127.6, 114.0, 55.29, 27.81, 25.54, 20.66, 18.08. IR (neat, cm⁻¹): 1804 (C═O), 1775 (C═O), 1732 (C═O). HRMS (ESI): Calcd. for C₁₅H₁₉N₂O₅ ([M+NH₄]⁺) m/z 307.12885. Found 307.12795. HPLC: Chiralcel OD-H (80 hexanes:20 isopropanol @ 0.8 ml/min): 95% ee; 27 min (minor) and 36 min (major).

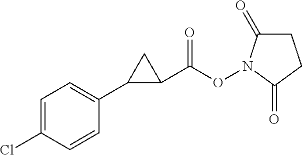

2,5-dioxopyrrolidin-1-yl 2-(4-chlorophenyl)cyclo-propanecarboxylate (1e) was obtained using the general procedure in 66% yield (48.9 mg). [α]²⁰_{D}=−279 (c=0.40, CHCl₃). ¹H NMR (400 MHz, CDCl₃): δ 7.25 (d, J=8.4 Hz, 2H), 7.05 (d, J=8.0 Hz, 2H), 2.81 (bs, 4H), 2.71-2.66 (m, 1H), 2.11-2.07 (m, 1H), 1.79-1.74 (m, 1H), 1.56-1.51 (m, 1H). ¹³C NMR (100 MHz, CDCl₃): δ 169.0, 169.4, 136.8, 132.8, 128.7, 127.7, 27.47, 25.54, 20.85, 18.22. IR (neat, cm⁻¹): 2980 (C—H), 2890 (C—H), 1802 (C═O), 1773 (C═O), 1730 (C═O). HRMS (ESI): Calcd. for C₁₄H₁₂ClNO₄Na ([M+Na]⁺) m/z 316.03471. Found 316.03380. HPLC: Chiralcel OD-H (80 hexanes:20 isopropanol @ 0.8 ml/min): 90% ee; 25 min (minor) and 32 min (major).

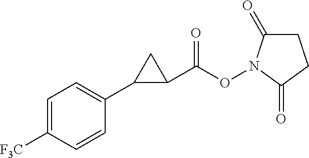

2,5-dioxopyrrolidin-1-yl 2-(4-(trifluoromethyl) phenyl) cyclopropanecarboxylate (1f) was obtained using the general procedure in 77% yield (63.6 mg). [α]²⁰_{D}=−226 (c=0.78, CHCl₃). ¹H NMR (400 MHz, CDCl₃): δ 7.54 (d, J=8.0 Hz, 2H), 7.22 (d, J=8.4 Hz, 2H), 2.81 (bs, 4H), 2.78-2.73 (m, 1H), 2.19-2.15 (m, 1H), 1.84-1.79 (m, 1H), 1.62-1.57 (m, 1H). ¹³C NMR (100 MHz, CDCl₃): δ 169.0, 168.3, 142.4, 129.5, 126.6, 125.58, 125.54. 27.52, 25.53, 21.07, 18.41. ¹⁹F NMR (376 MHz, CDCl₃): 8-62.96. IR (neat, cm⁻¹): 2978 (C—H), 2892 (C—H), 1802 (C═O), 1778 (C═O), 1737 (C═O). HRMS (ESI): Calcd. for C₁₅H₁₆F₃N₂O₄ ([M+NH₄]⁺) m/z 345.10567. Found 345.10450. HPLC: Chiralcel OD-H (80 hexanes:20 isopropanol @ 0.8 ml/min): 90% ee; 22 min (minor) and 26 min (major).

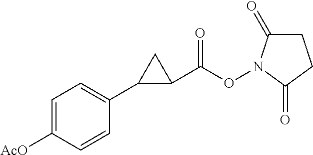

2,5-dioxopyrrolidin-1-yl 2-(4-acetoxyphenyl)cyclo-propanecarboxylate (1g) was obtained using the general procedure in 71% yield (56.8 mg). [α]20D=−224 (c=0.45, CHCl₃). ¹H NMR (400 MHz, CDCl₃): 7.16 (d, J=8.8 Hz, 2H), 7.03 (d, J=8.4 Hz, 2H), 2.83 (bs, 4H), 2.76-2.71 (m, 1H), 2.29 (s, 3H), 2.14-2.08 (m, 1H), 1.80-1.76 (m, 1H), 1.59-1.55 (m, 1H). ¹³C NMR (100 MHz, CDCl₃): 169.4, 169.0, 168.6, 149.6, 135.8, 127.5, 121.7, 27.67, 25.55, 21.06, 20.81, 18.26. IR (neat, cm⁻¹): 2963 (C—H), 1768 (C═O), 1735 (C═O). HRMS (ESI): Calcd. for C₁₆H₁₆NO₆ ([M+H]+) m/z 318.09721. Found 318.09737. HPLC: Chiralcel OD-H (80 hexanes:20 isopropanol @ 0.8 ml/min): 91% ee; 45 min (minor) and 57 min (major).

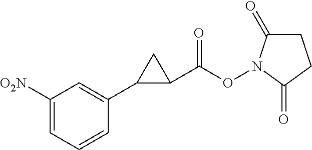

2,5-dioxopyrrolidin-1-yl 2-(3-nitrophenyl)cyclopropanecarboxylate (1h) was obtained using the general procedure in 50% yield (38.3 mg). [α]²⁰_{D}=−150 (c=0.19, CHCl₃). ¹H NMR (400 MHz, CDCl₃): δ 8.11-8.09 (m, 1H), 7.97 (s, 1H), 7.51-7.47 (m, 2H), 2.85-2.80 (m, 5H), 2.26-2.21 (m, 1H), 1.90-1.85 (m, 1H), 1.62-1.63 (m, 1H). ¹³C NMR (100 MHz, CDCl₃): δ 168.9, 168.1, 148.4, 140.5, 132.9, 129.6, 122.1, 121.0, 27.13, 25.54, 21.11, 18.33. IR (neat, cm⁻¹): 1808 (C═O), 1775 (C═O), 1731 (C═O), 1528 (NO₂), 1350 (NO₂). HRMS (ESI): Calcd. for C₁₄H₁₆N₃O₆ ([M+NH₄]⁺) m/z 322.10336. Found 322.10345. HPLC: Chiralcel AD-H (80 hexanes:20 isopropanol @ 0.8 ml/min): 92% ee; 40 min (minor) and 47 min (major).

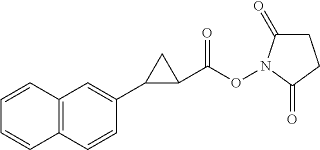

2,5-dioxopyrrolidin-1-yl 2-(naphthalen-2-yl)cyclopropanecarboxylate (11) was obtained using the general procedure in 33% yield (25.6 mg). [α]²⁰_{D}=−286 (c=1.06, CHCl₃). ¹H NMR (400 MHz, CDCl₃): δ 7.81-7.76 (m, 3H), 7.61 (s, 1H), 7.49-7.42 (m, 2H), 7.24-7.22 (m, 1H), 2.94-2.89 (m, 1H), 2.83 (bs, 4H), 2.26-2.22 (m, 1H), 1.88-1.83 (m, 1H), 1.74-1.69 (m, 1H). ¹³C NMR (100 MHz, CDCl₃): δ 169.1, 168.7, 135.6, 133.2, 132.5, 128.4, 127.6, 127.5, 126.4, 125.8, 125.2, 124.4, 28.48, 25.56, 20.82, 18.27. IR (neat, cm⁻¹): 2980 (C—H), 1802 (C═O), 1774 (C═O), 1739 (C═O). HRMS (ESI): Calcd. for C₁₈H₁₉N₂O₄ ([M+NH₄]⁺) m/z 327.13393. Found 327.13379. HPLC: Chiralcel AD-H (80 hexanes:20 isopropanol @ 0.8 ml/min): 91% ee; trans: 59 min (minor) and 92 min (major).

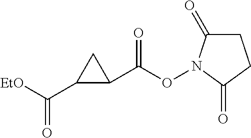

1-(2,5-dioxopyrrolidin-1-yl) 2-ethyl cyclopropane-1,2-dicarboxylate (1j) was obtained using the general procedure in 57% yield (36.6 mg). [α]²⁰_{D}=−235 (c=0.08, CHCl₃). [α]²⁰_{D}=−90 (c=3.8 mg/mL, CHCl₃). ¹H NMR (400 MHz, CDCl₃): δ 4.17 (q, J=7.2 Hz, 2H), 2.82 (bs, 4H), 2.45-2.41 (m, 1H), 2.37-2.33 (m, 1H), 1.67-1.57 (m, 2H), 1.28 (t, J=7.2 Hz, 3H). ¹³C NMR (100 MHz, CDCl₃): δ 170.5, 168.8, 167.5, 61.53, 25.50, 23.71, 19.05, 16.69, 14.07. IR (neat, cm⁻¹): 2984 (C—H), 1781 (C═O), 1727 (C═O). HRMS (ESI): Calcd. for C₁₁H₁₄NO₆ ([M+H]⁺) m/z 256.08156. Found 256.08129. HPLC: Chiralcel AD-H (80 hexanes:20 isopropanol @ 0.8 ml/min): 89% ee; 13 min (minor) and 16 min (major).

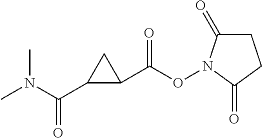

2,5-dioxopyrrolidin-1-yl 2-(dimethylcarbamoyl)cyclo-propanecarboxylate (1k) was obtained using the general procedure in 52% yield (33.4 mg). [α]20p=−101 (c=0.16, CHCl₃). ¹H NMR (400 MHz, CDCl₃): δ 3.17 (s, 3H), 2.97 (s, 3H), 2.82 (bs, 4H), 2.51-2.47 (m, 1H), 2.45-2.40 (m, 1H), 1.70-1.65 (m, 1H), 1.56-1.51 9 m, 1H). ¹³C NMR (100 MHz, CDCl₃): δ 168.9, 168.6, 168.4, 37.27, 35.96, 25.52, 22.66, 18.77, 16.31. IR (neat, cm⁻¹): 2924 (C—H), 2854 (C—H), 1782 (C═O), 1740 (C═O), 1637 (C═O). HRMS (ESI): Calcd. for C₁₁H₁₈N₃O₅ ([M+NH₄]⁺) m/z 272.12410. Found 272.12386. HPLC: Chiralcel AD-H (80 hexanes:20 isopropanol @ 0.8 ml/min): 96% ee; 19 min (minor) and 36 min (major).

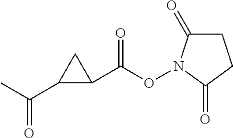

2,5-dioxopyrrolidin-1-yl 2-acetylcyclopropanecarboxylate (1l) was obtained using the general procedure in 55% yield (31.1 mg). [α]²⁰_{D}=−231 (c=0.25, CHCl₃). ¹H NMR (400 MHz, CDCl₃): δ 2.81 (bs, 4H), 2.65-2.60 (m, 1H), 2.45-2.41 (m, 1H), 2.35 (s, 3H), 1.60-1.56 (m, 2H). ¹³C NMR (100 MHz, CDCl₃): δ 203.9, 168.8, 167.6, 30.99, 30.37, 25.51, 20.63, 18.30. IR (neat, cm⁻¹): 2924 (C—H), 1780 (C═O), 1735 (C═O), 1704 (C═O). HRMS (ESI): Calcd. for C₁₀H₁₁NO₅Na ([M+Na]⁺) m/z 248.05294. Found 248.05239. HPLC: Chiralcel AD-H (80 hexanes:20 isopropanol @ 0.8 ml/min): 91% ee; 22 min (minor) and 39 min (major).

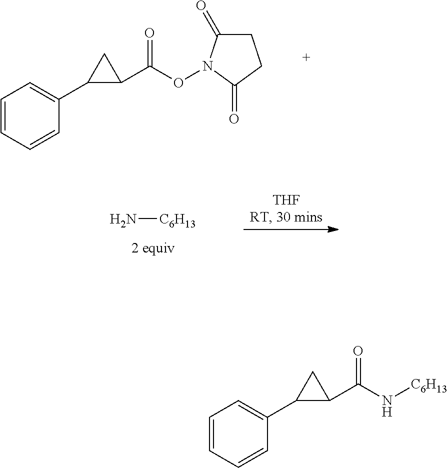

N-hexyl-2-phenylcyclopropanecarboxamide (2aa) was obtained in 92% yield (29.1 mg). [α]²⁰_{D}=−242 (c=0.49, CHCl₃). ¹H NMR (400 MHz, CDCl₃): δ 7.25-7.22 (m, 2H), 7.17-7.15 (m, 1H), 7.06-7.04 (m, 2H), 5.65 (bs, 1H), 3.26-3.22 (m, 2H), 2.45-2.43 (m, 1H), 1.58-1.51 (m, 2H), 1.47-1.45 (m, 2H), 1.26 (bs, 6H), 1.21-1.18 (m, 1H), 0.85 (m, 3H). ¹³C NMR (100 MHz, CDCl₃): δ 171.6, 140.9, 128.4, 126.1, 125.9, 39.86, 31.44, 29.64, 26.81, 26.57, 24.85, 22.51, 15.80, 13.97. IR (neat, cm⁻¹): 3295 (N—H), 2956 (C—H), 2925 (C—H), 2857 (C—H), 1634 (C═O). HRMS (ESI): Calcd. for C₁₆H₂₄NO ([M+H]⁺) m/z 246.1858. Found 246.1860. HPLC Chiralcel AD-H (80 hexanes:20 isopropanol @ 0.8 ml/min): 90% ee; 6 min (minor) and 8 min (major).

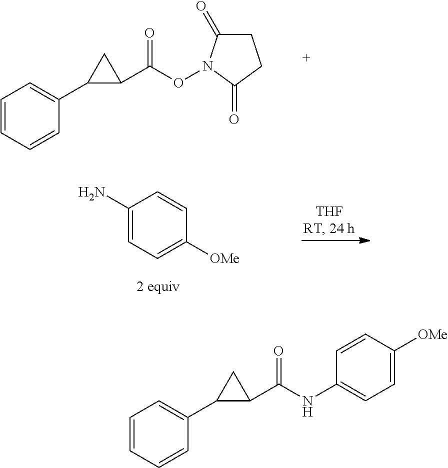

N-(4-methoxyphenyl)-2-phenylcyclopropanecarboxamide (2ab) was obtained in 62% yield (27.4 mg). [α]²⁰_{D}=−252 (c=0.54, CHCl₃). ¹H NMR (400 MHz, CDCl₃): δ 7.39 (d, J=8.0 Hz, 2H), 7.34 (bs, 1H), 7.29-7.24 (m, 2H), 7.20-7.19 (m, 1H), 7.09 (d, J=7.6 Hz, 2H), 6.82 (d, J=7.6 Hz, 2H), 3.76 (s, 3H), 2.55-2.54 (m, 1H), 1.70-1.68 (m, 2H), 1.32-1.30 (m, 1H). ¹³C NMR (100 MHz, CDCl₃): δ 170.2, 156.5, 140.8, 131.3, 128.7, 126.5, 126.2, 121.8, 114.3, 55.69, 27.74, 25.90, 16.46. IR (neat, cm⁻¹): 3274 (N—H), 2980 (C—H), 1643 (C═O). HRMS (ESI): Calcd. for C₁₇H₁₈NO₂ ([M+H]⁺) m/z 268.1337. Found 268.1337. HPLC Chiralcel AD-H (90 hexanes:10 isopropanol @ 1.0 ml/min): 89% ee; 16 min (major) and 28 min (minor).

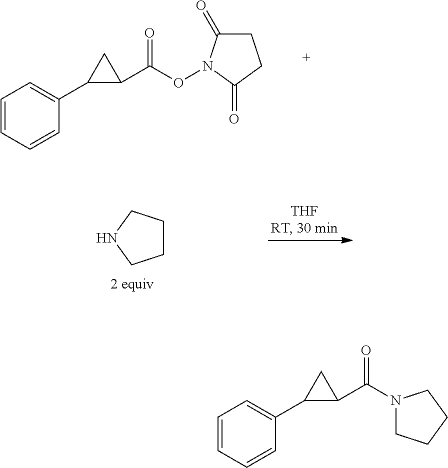

(2-phenylcyclopropyl)(pyrrolidin-1-yl)methanone (2ac) was obtained in 91% yield (32.4 mg). [α]²⁰_{D}=−376 (c=0.30, CHCl₃). ¹H NMR (400 MHz, CDCl₃): δ 7.27-7.23 (m, 2H), 7.18-7.14 (m, 1H), 7.11-7.09 (m, 2H), 3.61-3.53 (m, 2H), 3.48 (t, J=6.8 Hz, 2H), 2.52-2.47 (m, 1H), 1.97-1.81 (m, 5H), 1.65-1.61 (m, 1H), 1.25-1.21 (m, 1H). ¹³C NMR (100 MHz, CDCl₃): δ 170.4, 141.2, 128.3, 126.1 (2 Ar), 46.58, 46.01, 25.99, 25.39, 24.58, 24.40, 16.26. IR (neat, cm⁻¹): 2979 (C—H), 2873 (C—H), 1607 (C═O). HRMS (ESI): Calcd. for C₁₄H₁₈NO ([M+H]⁺) m/z 216.13829. Found 216.13775. HPLC Chiralcel OD-H (80 hexanes:20 isopropanol @ 0.8 ml/min): 92% ee; 7 min (minor) and 8 min (major).

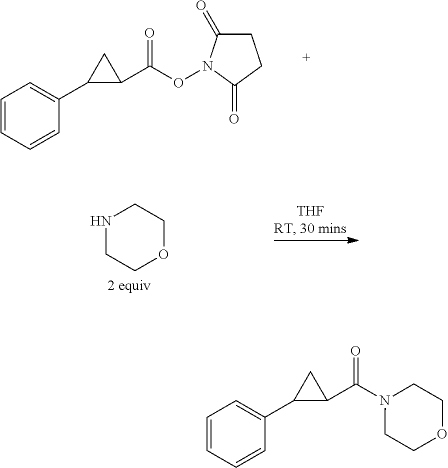

morpholino(2-phenylcyclopropyl)methanone (2ad) was obtained in 95% yield (40.9 mg). [α]²⁰_{D}=−178 (c=0.48, CHCl₃). ¹H NMR (400 MHz, CDCl₃): δ 7.28-7.25 (m, 2H), 7.20-7.16 (m, 1H), 7.10-7.08 (m, 2H), 3.66-3.61 (m, 8H), 2.50-2.45 (m, 1H), 1.93-1.89 (m, 1H), 1.69-1.63 (m, 1H), 1.30-1.24 (m, 1H). ¹³C NMR (100 MHz, CDCl₃): δ 170.6, 140.7, 128.4, 126.3, 125.9, 66.78 (2 C), 45.95, 42.53, 25.52, 22.91, 16.15. IR (neat, cm⁻¹): 2980 (C—H), 2890 (C—H), 1632 (C═O). HRMS (ESI): Calcd. for C₁₄H₁₈NO₂ ([M+H]⁺) m/z 232.13321. Found 232.13339. HPLC Chiralcel OD-H (80 hexanes:20 isopropanol @ 0.8 ml/min): 92% ee; 11 min (minor) and 17 min (major).

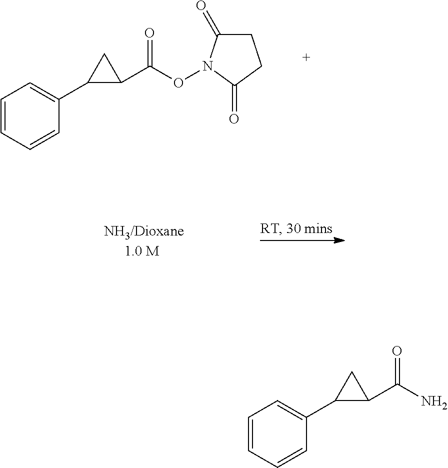

2-phenylcyclopropanecarboxamide (2ae) was obtained in 93% yield (29.6 mg). [α]²⁰_{D}=−290 (c=0.14, CHCl₃). ¹H NMR (400 MHz, CDCl₃): δ 7.25-7.22 (m, 2H), 7.19-7.16 (m, 1H), 7.08-7.05 (m, 2H), 5.82-5.71 (bd, 2H), 2.49-2.45 (m, 1H), 1.68-1.61 (m, 1H), 1.60-1.56 (m, 1H), 1.27-1.23 (m, 1H). ¹³C NMR (100 MHz, CDCl₃): δ 174.1, 140.5, 128.4, 126.3, 126.0, 25.83, 25.66, 16.29. IR (neat, cm⁻¹): 3382 (N—H), 3201 (N—H), 2922 (C—H), 1647 (C═O). HRMS (ESI): Calcd. for O₁₀H₁₂NO ([M+H]⁺) m/z 162.09134. Found 162.09066. HPLC Chiralcel OD-H (80 hexanes:20 isopropanol @ 0.8 ml/min): 94% ee; 8 min (major) and 10 min (minor).

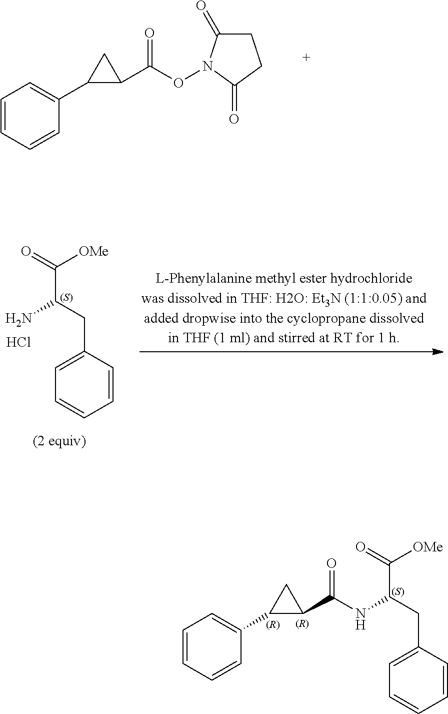

(2S)-methyl 3-phenyl-2-(2-phenylcyclopropane-carboxamido)propanoate (2af) was obtained in 66% yield (17.3 mg). [α]²⁰_{D}=−46 (c=0.34, CHCl₃). ¹H NMR (400 MHz, CDCl₃): δ 7.29-7.17 (m, 6H), 7.08-7.06 (m, 4H), 6.10 (d, J=7.6 Hz, 1H), 4.94-4.89 (m, 1H), 3.71 (s, 3H), 3.18-3.06 (m, 2H), 2.48-2.43 (m, 1H), 1.60-1.57 (m, 2H), 1.26-1.22 (m, 1H). ¹³C NMR (100 MHz, CDCl₃): δ 172.1, 171.4, 140.5, 135.7, 129.2, 128.5, 128.4, 127.1, 126.3, 126.1, 53.28, 52.30, 37.99, 26.38, 25.41, 15.82. IR (neat, cm⁻¹): 3312 (N—H), 3033 (C—H), 2950 (C—H), 1741 (C═O), 1639 (C═O). HRMS (ESI): Calcd. for C₂₀H₂₂NO₃ ({M+H]⁺) m/z 324.1599. Found 324.1597.

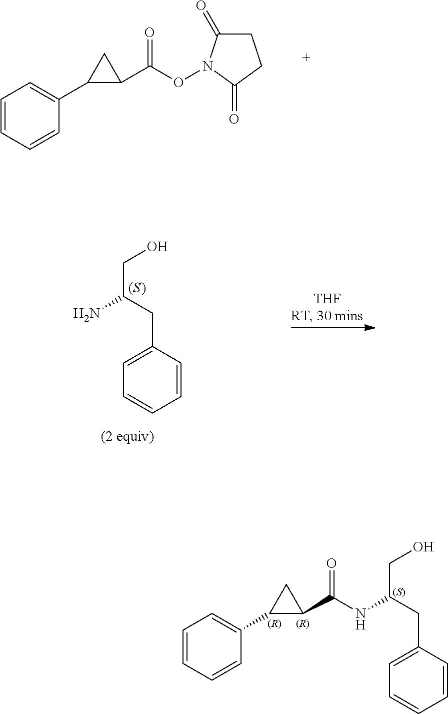

N—((S)-1-hydroxy-3-phenylpropan-2-yl)-2-phenylcyclo-propanecarboxamide (2ag) was obtained in 93% yield (20.8 mg). [α]²⁰_{D}=−161 (c=0.40, CHCl₃). ¹H NMR (400 MHz, CDCl₃): δ 7.29-7.16 (m, 8H), 7.05-7.03 (m, 2H), 6.00 (d, J=28.8 Hz, 1H), 4.19-4.17 (m, 1H), 3.68-3.66 (m, 1H), 3.59-3.55 (m, 1H), 2.91 (bs, 1H), 2.86 (d, J=7.2 Hz, 2H), 2.45-2.40 (m, 1H), 1.60-1.53 (m, 2H), 1.24-1.18 (m, 1H). ¹³C NMR (100 MHz, CDCl₃): δ 172.5, 140.5, 137.5, 129.2, 128.6, 128.4, 126.6, 126.2, 126.0, 64.01, 53.14, 37.07, 26.63, 25.16, 15.94. IR (neat, cm⁻¹): 3284 (O—H, N—H), 2954 (C—H), 1633 (C═O). HRMS (ESI): Calcd. for C₁₉H₂₂NO₂ ([M+H]⁺) m/z 296.1650. Found 296.1645.

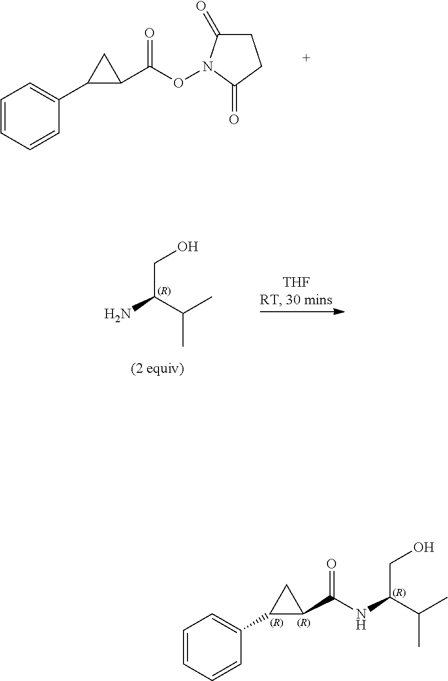

N—((R)-1-hydroxy-3-methylbutan-2-yl)-2-phenylcyclo-propanecarboxamide (2ah) was obtained in 54% yield (11.2 mg). [α]²⁰_{D}=−156 (c=0.22, CHCl₃). ¹H NMR (400 MHz, CDCl₃): δ 7.27-7.23 (m, 2H), 7.19-7.17 (m, 1H), 7.07-7.05 (m, 2H), 5.87 (d, J=7.2 Hz, 1H), 3.74-3.72 (m, 1H), 3.65-3.64 (m, 2H), 2.78 (bs, 1H), 2.47-2.44 (m, 1H), 1.63-1.60 (m, 2H), 1.24-1.21 (m, 1H), 0.945 (m, 6H). ¹³C NMR (100 MHz, CDCl₃): δ 172.9, 140.7, 128.4, 126.2, 125.9, 64.21, 57.56, 29.13, 26.78, 25.21, 19.43, 18.75, 16.17. IR (neat, cm⁻¹): 3287 (O—H, N—H), 2924 (C—H), 1637 (C═O). HRMS (ESI): Calcd. for C₁₅H₂₂NO₂ ({M+H]⁺) m/z 248.1650. Found 248.1657.

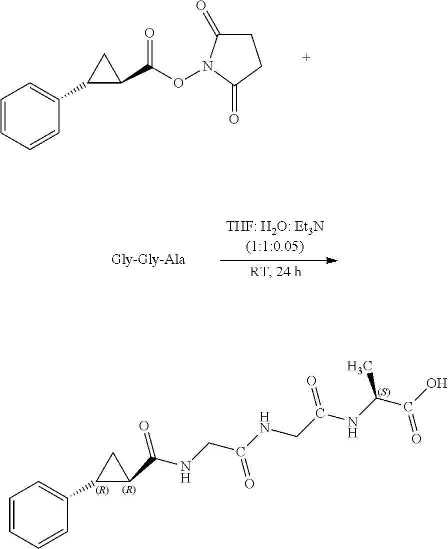

2-(2-(2-(2-phenylcyclopropanecarboxamido)-acetamido)acetamido)propanoic acid² (2ai) was obtained in 60% yield (30.8 mg). The cyclopropyl peptide was purified by preparatory HPLC using a Dionex Summit HPLC equipped with the Waters Radial Compression column (300 mm×25 mm, 15 micron particle size, 300 Angstrom pore size, C4) utilizing a gradient solvent system of acetonitrile and water (5% MeCN/H₂0-50% MeCN/H₂O) with a flow rate of 20 ml/min. [α]²⁰_{D}=−185 (c=0.19, IPA). ¹H NMR (400 MHz, D₂O): δ 7.24-7.20 (m, 2H), 7.15-7.12 (m, 1H), 7.09-7.08 (m, 2H), 4.24 (t, J=7.6 Hz, 1H), 3.84-3.82 (m, 4H), 2.36-2.31 (m, 1H), 1.88-1.83 (m, 1H), 1.42-1.37 (m, 1H), 1.32-1.25 (m, 4H). ¹³C NMR (100 MHz, D₂O): δ 179.4, 178.9, 175.1, 173.7, 143.1, 131.4, 129.3, 128.8, 51.55, 45.67, 44.86, 28.26, 27.69, 18.95, 18.32. IR (neat, cm⁻¹): 3303 (O—H, N—H), 2979 (C—H), 1651 (C═O), 1635 (C═O). HRMS (ESI): Calcd. for C₁₇H₂₂N₃O₅ ([M+H]⁺) m/z 348.1559. Found 348.1550. Triethylamine remains as an impurity as seen by residual solvent peaks in the ¹H NMR and ¹³C NMR. The amine and carboxylic acid protons were not observed in D₂O.

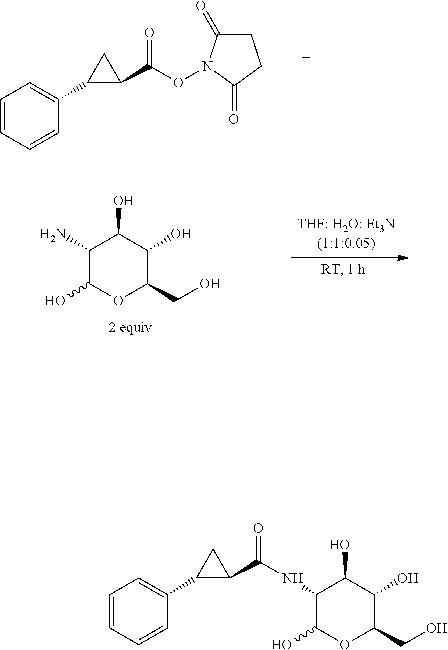

N-(2-phenylcyclopropanecarboxamido)-D-glucosamine (2aj) was obtained in 47% yield (20.4 mg) as a mixture of anomers (α:β=1.6:1) as determined by HPLC. The product was purified by preparatory HPLC using a Dionex Summit HPLC equipped with the Supelcosil PLC-8 column (250 mm×21.2 mm, 12 micron particle size, C8) utilizing a gradient solvent system of acetonitrile in water (5% MeCN:H₂O-30% MeCN:H₂O) with a flow rate of 20 ml/min. [α]²⁰_{D}=−51 (c=0.78, DMSO). ¹H NMR (400 MHz, DMSO) (anomeric mixture α:β=2:1): δ 8.02 (d, J=8.4 Hz, 1H, β anomer), 7.95 (d, J=8.0 Hz, 1H, α anomer), 7.29-7.25 (m, 2H, anomeric mixture), 7.18-7.14 (m, 1H, anomeric mixture), 7.10-7.09 (m, 2H, anomeric mixture), 6.52 (d, J=6.4 Hz, 1H, β anomer), 6.41 (d, J=4.4 Hz, 1H, α anomer), 4.94-4.92 (m, 1H, mixture of anomers), 4.88 (d, J=5.6 Hz, 1H, α anomer), 4.85 (d, J=5.6 Hz, 1H, β anomer), 4.65 (d, J=5.6 Hz, 1H, α anomer), 4.50 (t, J=5.6 Hz, 1H, β anomer), 4.45-4.40 (m, 1H), 3.69-3.56 (m, 2H, anomeric mixture), 3.53-3.30 (m, 3H, anomeric mixture), 3.27 (m, 1H, β anomer), 3.11 (m, 1H, α anomer), 3.05 (m, 1H, anomeric mixture), 2.26-2.18 (m, 1H, anomeric mixture), 2.13-2.09 (m, 1H, a anomer), 1.89-1.85 (m, 1H, β anomer), 1.36-1.30 (m, 1H, anomeric mixture), 1.20-1.10 (m, 1H, anomeric mixture). ¹³C NMR (100 MHz, DMSO) (α anomer): δ 171.1, 141.3, 128.3 (2), 125.8, 125.7 (2), 90.58, 72.05, 71.22, 70.50, 61.14, 54.47, 25.32, 23.83, 15.59. ¹³C NMR (100 MHz, DMSO) (β anomer): δ 171.13, 141.3, 128.3 (2), 125.8, 125.7 (2), 95.49, 76.77, 74.45, 70.88, 61.14, 57.39, 26.11, 23.83, 15.59. IR (neat, cm⁻¹): 3285 (O—H, N—H), 1637 (C═O), 1613 (C═O), 1563 (C═C). HRMS (ESI): Calcd. for C₁₆H₂₂NO₆ ([M+H]⁺) m/z 324.1447. Found 324.1469.

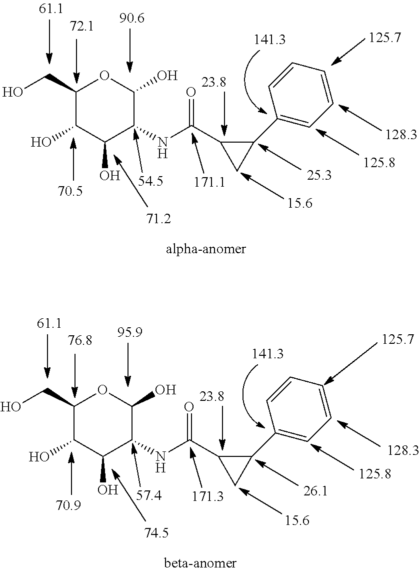

What is claimed is:

1. A composition comprising a stereoisomer corresponding to Formula CA-2, the stereoisomer having an enantiomer and the composition having an enantiomeric excess of the sterioisomer corresponding to Formula CA-1 over the enantiomer: 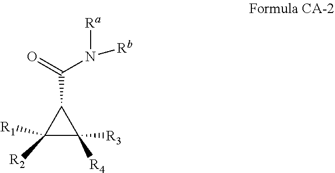
wherein R₁, R₂, R₃ and R₄ are independently hydrogen, hydrocarbyl, substituted hydrocarbyl, heterocyclo, or EWG, and each EWG is independently an electron withdrawing group, and R^{a} and R^{b} are independently hydrogen, hydrocarbyl, substituted hydrocarbyl, or heterocyclo.

2. The composition of claim 1 wherein the enantiomeric excess is greater than 90%.

3. The composition of claim 2 wherein R^{a} is hydrogen and R^{b} is the residue of or comprises a naturally occurring or synthetic α, β, γ, or Δ amino acid or a sugar.

4. The composition of claim 2 wherein R^{a} is hydrogen and R^{b} is or comprises a polypeptide or a polysaccaride.

5. The composition of claim 2 wherein R^{a} is hydrogen and R^{b} is or comprises an amino sugar or a nucleotide sugar.

6. The composition of claim 1 wherein R^{a} is hydrogen and R^{b} is the residue of or comprises a naturally occurring or synthetic α, β, γ, or Δ amino acid or a sugar.

7. The composition of claim 1 wherein R^{a} is hydrogen and R^{b} is or comprises a polypeptide or a polysaccaride.

8. The composition of claim 1 wherein R^{a} is hydrogen and R^{b} is or comprises an amino sugar or a nucleotide sugar.

9. The composition of claim 1 wherein the stereoisomer corresponds to Formula CA-6: 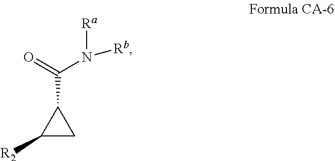
and the enantiomeric excess is greater than 90%.

10. The composition of claim 10 wherein R^{a} is hydrogen and R^{b} is the residue of or comprises a naturally occurring or synthetic α, β, γ, or Δ amino acid or a sugar.

11. A process for the preparation of a chiral cyclopropyl carboxamide in enantioenriched form, the process comprising treating a succinimidyl cyclopropyl carboxylate with an amine.

12. The process of claim 11 wherein the succinimidyl cyclopropyl carboxylate corresponds to Formula C1 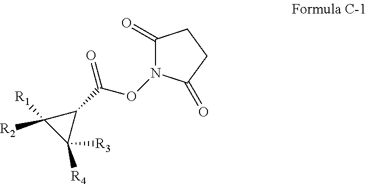
R₁, R₂, R₃ and R₄ are independently hydrogen, hydrocarbyl, substituted hydrocarbyl, heterocyclo, or EWG, and each EWG is independently an electron withdrawing group.

13. The process of claim 12 wherein the amine has the formula HNR^{a}R^{b} and R^{a} and R^{b} are independently hydrogen, hydrocarbyl, substituted hydrocarbyl, or heterocyclo.

14. The process of claim 11 wherein the amine has the formula HNR^{a}R^{b} and R^{a} and R^{b} are independently hydrogen, hydrocarbyl, substituted hydrocarbyl, or heterocyclo.

15. The process of claim 11 wherein the process further comprises the step of treating an olefin with succinimidyl diazoacetate in the presence of a metal porphyrin complex to form the succinimidyl cyclopropyl carboxylate.

16. The process of claim 15 wherein olefin corresponds to Formula O-1 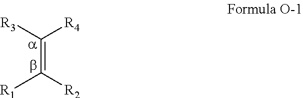
R₁, R₂, R₃ and R₄ are independently hydrogen, hydrocarbyl, substituted hydrocarbyl, heterocyclo, or EWG, and each EWG is independently an electron withdrawing group.

17. The process of claim 12 wherein the process further comprises the step of treating an olefin with succinimidyl diazoacetate in the presence of a metal porphyrin complex to form the succinimidyl cyclopropyl carboxylate.

18. The process of claim 17 wherein olefin corresponds to Formula O-1 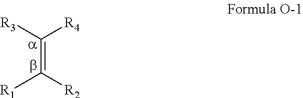
R₁, R₂, R₃ and R₄ are independently hydrogen, hydrocarbyl, substituted hydrocarbyl, heterocyclo, or EWG, and each EWG is independently an electron withdrawing group.

19. The process of claim 13 wherein the process further comprises the step of treating an olefin with succinimidyl diazoacetate in the presence of a metal porphyrin complex to form the succinimidyl cyclopropyl carboxylate.

20. The process of claim 19 wherein olefin corresponds to Formula O-1 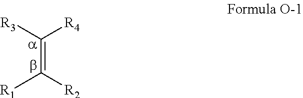
R₁, R₂, R₃ and R₄ are independently hydrogen, hydrocarbyl, substituted hydrocarbyl, heterocyclo, or EWG, and each EWG is independently an electron withdrawing group.

21. The process of claim 14 wherein the process further comprises the step of treating an olefin with succinimidyl diazoacetate in the presence of a metal porphyrin complex to form the succinimidyl cyclopropyl carboxylate.

22. The process of claim 21 wherein olefin corresponds to Formula O-1 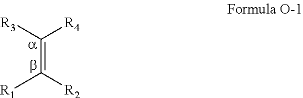
R₁, R₂, R₃ and R₄ are independently hydrogen, hydrocarbyl, substituted hydrocarbyl, heterocyclo, or EWG, and each EWG is independently an electron withdrawing group.

23. The process of claim 15 wherein the metal porphyrin complex is selected from the group consisting of cobalt porphyrin complexes corresponding to formula [Co(P1)], [Co(P2)], and [Co(P3)]:

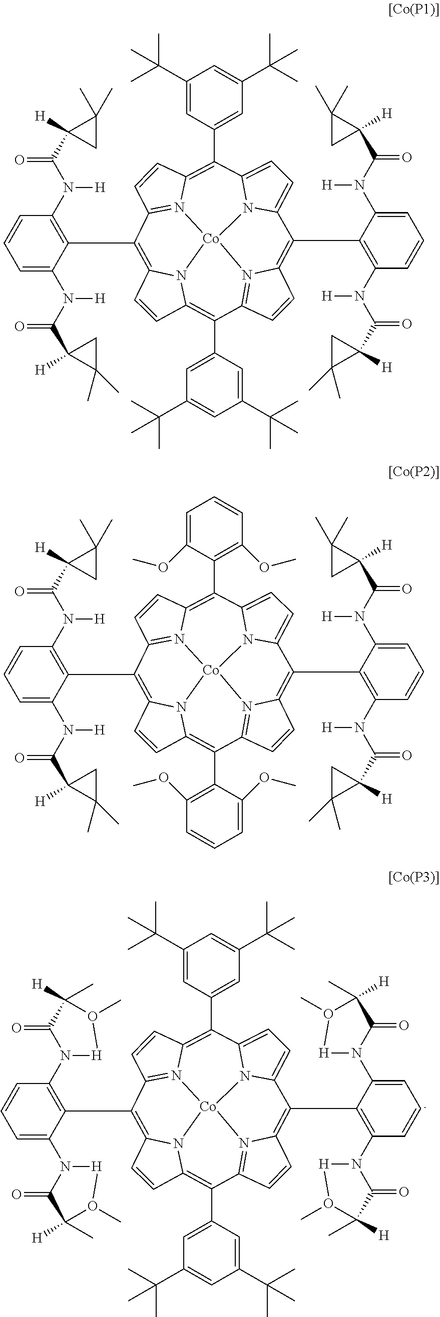

24. The process of claim 17 wherein the metal porphyrin complex is selected from the group consisting of cobalt porphyrin complexes corresponding to formula [Co(P1)], [Co(P2)], and [Co(P3)]:

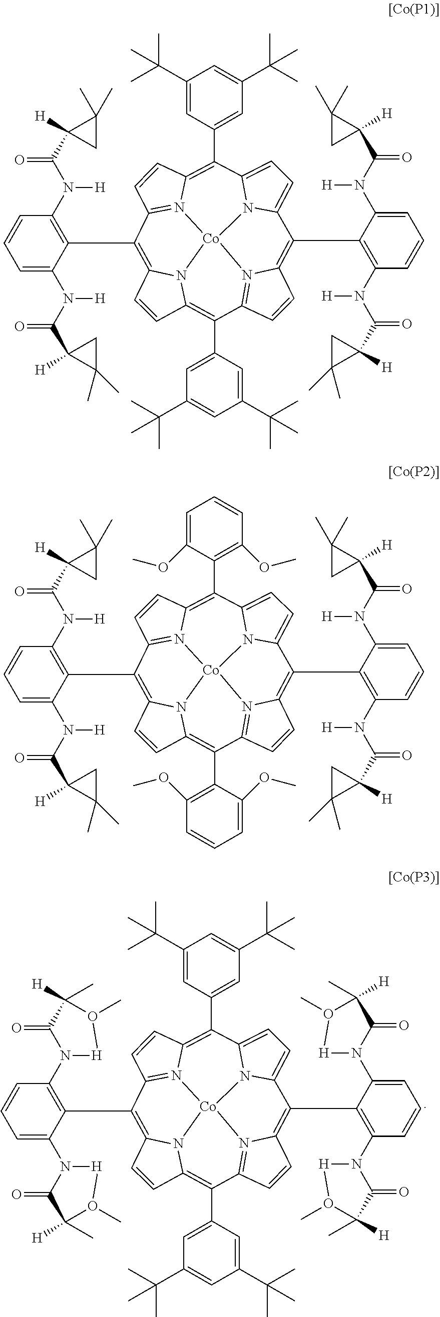

25. The process of claim 19 wherein the metal porphyrin complex is selected from the group consisting of cobalt porphyrin complexes corresponding to formula [Co(P1)], [Co(P2)], and [Co(P3)]:

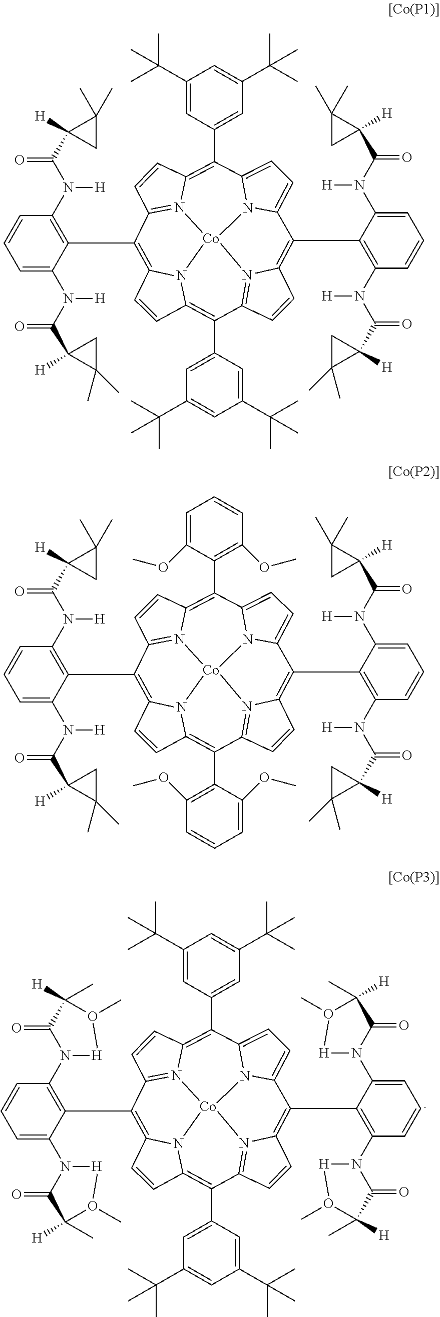

26. A process for the preparation of a chiral cyclopropyl carboxamide in enantioenriched form, the process comprising treating a stereoisomer with an amine in a reaction mixture, the stereoisomer having an enantiomer and the reaction mixture having an enantiomeric excess of the sterioisomer wherein,
the stereoisomer corresponds to Formula C-1 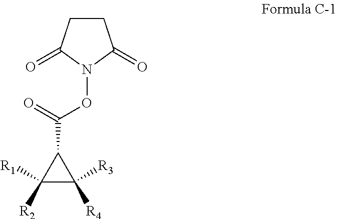
R₁, R₂, R₃ and R₄ are independently hydrogen, hydrocarbyl, substituted hydrocarbyl, heterocyclo, or EWG, and
each EWG is independently an electron withdrawing group.

27. The process of claim 26 wherein the stereoisomer corresponds to Formula C-2 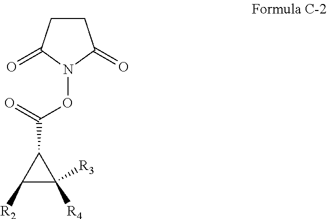
R₂ is hydrocarbyl, substituted hydrocarbyl, heterocyclo, or EWG,
R₃ and R₄ are independently hydrogen, hydrocarbyl, substituted hydrocarbyl, heterocyclo, or EWG and
each EWG is independently an electron withdrawing group.

28. The process of claim 27 wherein R₃ and R₄ are hydrogen.

29. The process of claim 27 wherein the metal porphyrin complex is selected from the group consisting of cobalt porphyrin complexes corresponding to formula [Co(P1)], [Co(P2)], and [Co(P3)]:

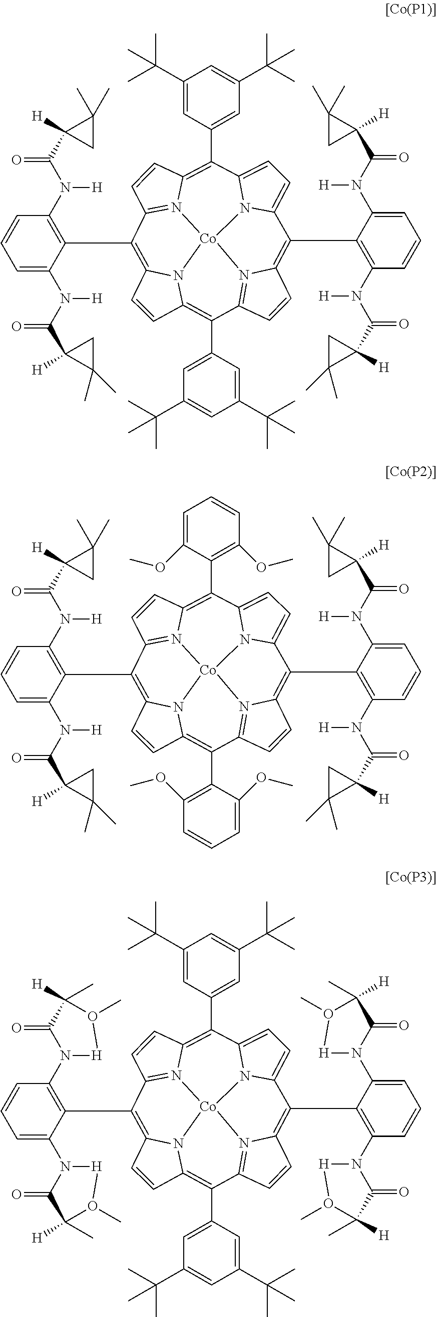

30. The process of claim 27 wherein the amine has the formula HNR^{a}R^{b}, and R^{a} and R^{b} are independently hydrogen, hydrocarbyl, substituted hydrocarbyl, or heterocyclo.

31. The process of claim 27 wherein the amine has the formula HNR^{a}R^{b}, R^{a} and R^{b} are independently hydrogen, hydrocarbyl, substituted hydrocarbyl, or heterocyclo and the metal porphyrin complex is selected from the group consisting of cobalt porphyrin complexes corresponding to formula [Co(P1)], [Co(P2)], and [Co(P3)]:

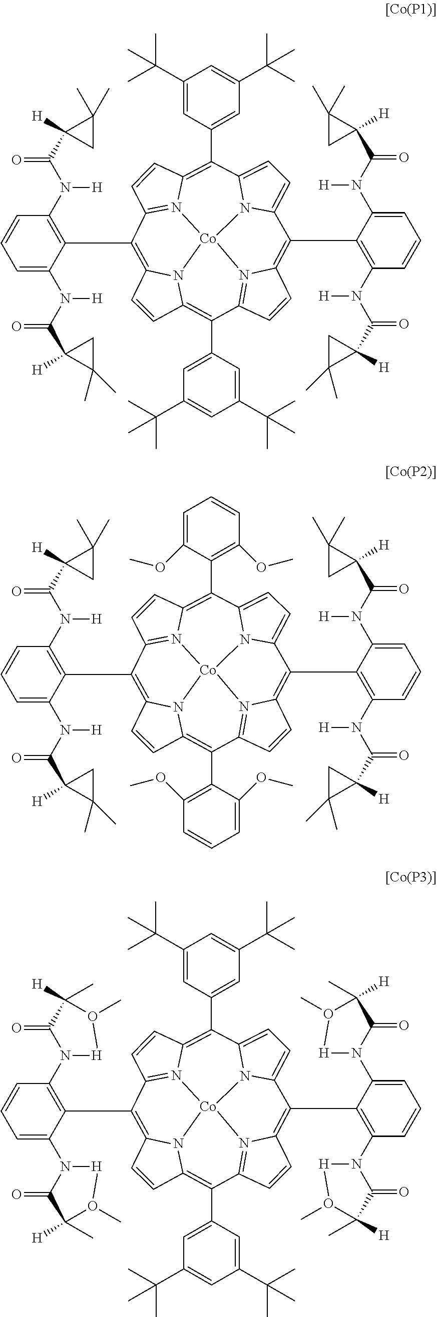

32. A succinimidyl cyclopropyl carboxylate corresponding to Formula C 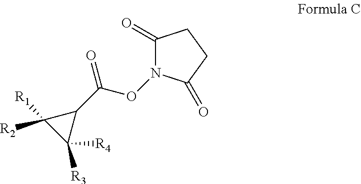
wherein R₁, R₂, R₃ and R₄ are independently hydrogen, hydrocarbyl, substituted hydrocarbyl, heterocyclo, or EWG, and each EWG is independently an electron withdrawing group.

33. A composition comprising a stereoisomer corresponding to Formula C-1, the stereoisomer having an enantiomer and the composition having an enantiomeric excess of the sterioisomer corresponding to Formula C-1 over the enantiomer: 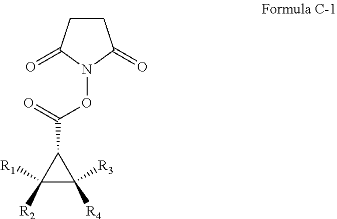
wherein R₁, R₂, R₃ and R₄ are independently hydrogen, hydrocarbyl, substituted hydrocarbyl, heterocyclo, or EWG, and each EWG is independently an electron withdrawing group.

34. The composition of claim 33 wherein the stereoisomer corresponds to Formula C-2 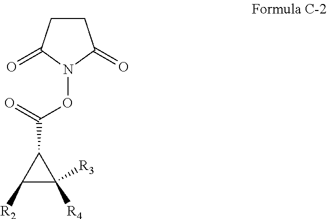
R₂, is hydrocarbyl, substituted hydrocarbyl, heterocyclo, or EWG,
R₃ and R₄ are independently hydrogen, hydrocarbyl, substituted hydrocarbyl, heterocyclo, or EWG and
each EWG is independently an electron withdrawing group.

35. The composition of claim 34 wherein R₃ and R₄ are hydrogen.

36. The composition of claim 33 wherein the enantiomeric excess is greater than 90%.

37. The composition of claim 34 wherein the enantiomeric excess is greater than 90%.
